(19) United States
(12) Patent Application Publication
(10) Pub. No.: US 2023/0055519 A1
(43) Pub. Date: Feb. 23, 2023

(54) METHODS OF IDENTIFYING SYNTHETIC MOLECULAR BINDING AGENTS

(71) Applicants: THE TRANSLATIONAL GENOMICS RESEARCH INSTITUTE, Phoenix, AZ (US); ARIZONA BOARD OF REGENTS ON BEHALF OF NORTHERN ARIZONA UNIVERSITY, Flagstaff, AZ (US)

(72) Inventors: Paul Keim, Flagstaff, AZ (US); Erik Settles, Flagstaff, AZ (US); Jason Ladner, Flagstaff, AZ (US); John Altin, Phoenix, AZ (US); Charles Hall Davis Williamson, IV, Flagstaff, AZ (US); Sunil Sharma, Phoenix, AZ (US)

(21) Appl. No.: 17/793,383

(22) PCT Filed: Jan. 16, 2021
(86) PCT No.: PCT/US2021/013774

§ 371 Date: Jul. 15, 2022

Related U.S. Application Data
(60) Provisional application No. 62/961,930, filed on Jan. 16, 2020.

(51) Int. Cl. C40B 30/04; C12N 15/10; C12Q 1/6809; C12N 15/62

(52) U.S. Cl. CPC C40B 30/04; C12N 15/1058; C12Q 1/6809; C12N 15/62

(57) ABSTRACT

The present invention provides methods of maturing a peptide library and of identifying peptides having increased specific binding to a target molecule and/or differential specific binding to target molecules. The present invention also provide methods of developing diagnostic assays, detection kits and therapeutic agents with the peptides.

### CROSS REFERENCE TO RELATED APPLICATIONS

This application claims the benefit of U.S. Provisional Patent Application No. 62/961,930, filed on Jan. 16, 2020, the contents of which are incorporated herein by reference in its entirety.

### TECHNICAL FIELD

This application relates to a method for identifying synthetic molecular binding agents from peptide libraries. In some implementations, this method uses large libraries of peptides tagged with DNA sequences to precisely identify particular peptides. Target molecules (e.g., proteins, toxins, enzymes, pathogens, biomarkers) are incubated with the libraries to identify specific peptides that bind to the targets. The candidate peptides are used to design subsequent libraries to explore their chemical derivatives and identify better binding agents. The best binding agents are used as the basis for detectors, diagnostics, and potential therapeutic agents.

### BACKGROUND

Molecules that bind to critical targets (e.g., organisms, proteins, toxins, or other biological molecules) have great potential as diagnostic/detection tools and also as potential therapeutic drugs. The current approach is to use large synthetic small-molecule libraries that have to be individually chemically synthesized. The current approach is labor intensive and lacks in cost- and time-effectiveness.

A methodology is needed that would be amenable to abiotic synthetic processes under a quality-controlled manufacturing system. The generation of synthetic molecular binding agents (SYMBAs) requires the ability to explore a very large number of highly diverse molecules in order to identify rare high affinity binding agents. One of the obstacles to exploring the binding affinities of such large numbers of molecules is the ability to account for slight differences in the starting abundances of the molecules in the context of identifying, from the very large number of molecules, which ones have measurable specific affinities to the critical target. Accordingly, it would also be desirable for the new methodology to efficiently determine and evaluate binding interactions from a very large number of molecules.

### SUMMARY

The present invention relates to methods for identifying synthetic molecular binding agents (SYMBA) from peptide libraries. This method uses large libraries of peptides, which in some embodiments are tagged with DNA sequences to precisely identify particular peptides. The peptide libraries comprise at least 300 peptide constructs, for example, at least 500 peptide constructs or at least 1,000 peptide constructs. Preferably, the peptide libraries comprise at least 100,000, at least 150,000, at least 200,000, or at least 250,000 peptide constructs. In some aspects, the peptide libraries comprise a plurality of negative controls. Target molecules (which may be proteins, toxins, enzymes, pathogens, cells, or biomarkers) are incubated with the peptide libraries to identify specific peptides that bind to the targets. The candidate peptides are used to design subsequent libraries to explore their chemical derivatives and identify better binding agents. The best binding agents are used as the basis for detectors, diagnostics, and potential therapeutic agents. The binding assays performed in the disclosed methods comprise a plurality of negative controls. The disclosure also relates to the methods of maturing a peptide library to improve binding to a target molecule.

The disclosed methods allow the computational design of diverse molecular libraries and the high-capacity screening of them. The binding maturation cycles that follow identify superior binding agents in a directed design, rapid and economical strategy.

The methods of maturing a peptide library to improve binding to a target molecule comprise identifying a first peptide having specific binding to the target molecule and having an identified threshold z-score and generating a library of peptide constructs based on the first peptide. The library of peptide constructs comprises a peptide construct comprising the first peptide and a plurality of peptide constructs comprising variant peptides. The method further comprises contacting the target molecule with the library of peptide and identifying at least one variant peptide with increased binding to the target molecule compared to that of the first peptide. Increased binding is indicated by a z-score higher than the identified threshold z-score of the first peptide. The z-score of a peptide is calculated by first determining a relative abundance level of each peptide constructs in the library of peptide constructs and then grouping the grouping peptide constructs into bins based on similarity of relative abundance level, wherein each bin comprises at least 300 peptide constructs. The relative abundance level of each peptide construct is also normalized against the average of the relative abundance level of the negative control peptide constructs in the library of peptide constructs. The normalized relative abundance levels of each peptide construct in a bin are used to determine a mean and a standard deviation of each bin. The z-score of a peptide is calculated based on the mean and a standard deviation of its bin. In some aspects, the determination of the mean and the standard deviation of the normalized relative abundance levels in a bin excludes peptide constructs having outlier relative abundance levels. In some aspects, a peptide construct has an outlier relative abundance level when its normalized relative abundance level is outside the 95% highest density interval of its bin. In certain implementations, 5% of peptide constructs in each bin are excluded from the determination of the mean and the standard deviation of the normalized relative abundance levels in a bin.

In some aspects, the variant peptides of the plurality of peptide constructs are produced by complete single residue mutagensis. Such variant peptide differs from the first peptide by a single point mutation, which is a substitution of the original amino acid with the nineteen other amino acids. Thus, the plurality of peptide constructs comprises nineteen different variants peptides for each substituted residue of the first peptide. In other aspects, the variant peptides of the plurality of peptide constructs are created by sliding window mutagenesis. Thus, each variant peptide differs from the first peptide by at least two contiguous residues from either the C-terminus end or the N-terminus end of the first peptide. In still other aspects, the variant peptides of the plurality of peptide constructs are produced by alanine scanning mutagenesis. In one aspect, these variant peptides differ from the first peptide by a single point mutation and each point mutation is a substitution with alanine. In another aspect, each point mutation is a substitution with glycine. In certain implementations, the plurality of variant peptides comprises at least one of the sets of variant peptides produce by complete single residue mutagenesis, sliding window mutagenesis, and alanine scanning mutagenesis. In some aspects, the first peptide comprises a consensus sequence generated from bound peptides. Accordingly, in some embodiments, the plurality of peptide constructs comprises variant constructs comprising a core sequence having at least 5 consecutive amino acids from the consensus sequence. In particular embodiments, at least two variant peptides are identified from the library of peptide constructs with increased binding to the target molecule compared to that of the first peptide. Such methods further comprise generating a second library of peptide constructs that comprises multimers of the at least two variant peptides. In some aspects, the multimers are dimers, for example a heterodimer formed by two variant peptides identified to have increased binding to the target molecule compared to that of the first peptide.

In some embodiments, the methods of maturing a peptide library to improve binding to a target molecule further comprises generating a second library of peptide constructs, contacting the target molecule with the second library of peptide constructs, and identifying at least one variant peptide from the second library of peptide constructs with increased binding to the target molecule compared to that of the second peptide. The second library of peptide constructs is based on a second peptide identified as having increased binding to the target molecule compared to that of the first peptide, for example by having a z-score higher than the threshold z-score of the first pepetide. Thus, the second library of peptide constructs comprises a peptide construct comprising the second peptide and a second plurality of peptide constructs comprising variant peptides. In some implementations, the second plurality of peptide constructs comprises variant peptides produced by alanine scanning mutagenesis. Accordingly, the variant peptides differ from the second peptide by a single point mutation and each point mutation is a substitution with alanine or glycine. The at least one variant peptide from the second library of peptide constructs with increased binding to the target molecule compared to that of the second peptide has a higher z-score than the z-score of the second peptide.

The methods of identifying a peptide with increased specific binding to a target molecule comprise providing a first library of peptide constructs and contacting the target molecule with the first library of peptide constructs in a first binding assay to produce at least one peptide construct of the first library of peptide constructs bound to the target molecule and at least one peptide construct of the first library of peptide constructs not bound to the target molecule. The z-score of at least one peptide construct of the first library of peptide constructs not bound to the target molecule is less than a z-score of at least one peptide construct of the first library of peptide constructs bound to the target molecule. The z-score of a peptide is calculated by first determining a relative abundance level of each peptide constructs in the library of peptide constructs and then grouping the grouping peptide constructs into bins based on similarity of relative abundance level, wherein each bin comprises at least 300 peptide constructs. The relative abundance level of each peptide construct is also normalized against the average of the relative abundance level of the negative control peptide constructs in the library of peptide constructs. The normalized relative abundance levels of each peptide construct in a bin are used to determine a mean and a standard deviation of each bin. The z-score of a peptide is calculated based on the mean and a standard deviation of its bin. In some aspects, the determination of the mean and the standard deviation of the normalized relative abundance levels in a bin excludes peptide constructs having outlier relative abundance levels. In some aspects, a peptide construct has an outlier relative abundance level when its normalized relative abundance level is outside the 95% highest density interval of its bin. In certain implementations, 5% of peptide constructs in each bin are excluded from the determination of the mean and the standard deviation of the normalized relative abundance levels in a bin.

The methods of identifying a peptide with increased specific binding to a target molecule further comprise identifying a first peptide from the at least one peptide construct of the first library of peptide constructs bound the target molecule, generating a second library of peptide constructs based on the first pepetide to identify a higher affinity peptide, and identifying at least one variant peptide from the second library of peptide constructs with increased binding to the target molecule compared to that of the first peptide. The z-score of the first peptide from the first binding assay is a threshold z-score. Thus, the z-score of the identified at least one variant peptide with increased binding to the target molecule has a higher z-score than the threshold z-score. In some implementations, the methods comprise separating the at least one peptide construct of the first library of peptide constructs bound the target molecule from the at least one peptide construct of the first library of peptide constructs not bound to the target molecule.

The second library of peptide constructs comprises a peptide construct comprising the first peptide and a plurality of peptide constructs comprising variant peptides. In some aspects, the variant peptides of the plurality of peptide constructs are produced by complete single residue mutagensis. Such variant peptide differs from the first peptide by a single point mutation, which is a substitution of the original amino acid with the nineteen other amino acids. Thus, the plurality of peptide constructs comprises nineteen different variants peptides for each substituted residue of the first peptide. In other aspects, the variant peptides of the plurality of peptide constructs are created by sliding window mutagenesis. Thus, each variant peptide differs from the first peptide by at least two contiguous residues from either the C-terminus end or the N-terminus end of the first peptide. In still other aspects, the variant peptides of the plurality of peptide constructs are produced by alanine scanning mutagenesis. In one aspect, these variant peptides differ from the first peptide by a single point mutation and each point mutation is a substitution with alanine. In another aspect, each point mutation is a substitution with glycine. In certain implementations, the plurality of variant peptides comprises at least one of sets of variant peptide produce by complete single residue mutagenesis, sliding window mutagenesis, and alanine scanning mutagenesis. The plurality of peptide constructs may also comprise variant peptides that comprises at least five consecutive amino acids from the first peptide and at least one of the five consecutive amino acids in the variant peptide construct is substituted with a different amino acid. In some aspects, the first peptide comprises a consensus sequence generated from bound peptides of the first binding assay. Accordingly, in some embodiments, the plurality of peptide constructs comprises variant constructs comprising a core sequence having at least 5 consecutive amino acids from the consensus sequence.

In particular embodiments, at least two variant peptides are selected from the first library of peptide constructs with increased binding to the target molecule compared to that of the first peptide. Thus, the second library of peptide constructs generated may comprises multimers of the at least two variant peptides. In some aspects, the multimers are dimers, for example a heterodimer formed by two variant peptides identified to have increased binding to the target molecule compared to that of the first peptide. In such implementations, the threshold z-score is the highest z-score of the selected peptides.

Also described are methods of identifying a peptide with differential specific binding to the first target molecule and a second target molecule. The methods comprise providing a first library of peptide constructs and contacting the first library of peptide constructs with the first target molecule in a first binding assay and with the second target molecule in a second binding assay. The first and the second binding assays produce at least one peptide construct of the first library of peptide constructs bound to the first target molecule, at least one peptide construct of the first library of peptide constructs not bound to the target molecule, at least one peptide construct of the first library of peptide constructs bound to the second target molecule, and at least one peptide construct of the first library of peptide constructs not bound to the second target molecule. The z-score of at least one peptide construct of the first library of peptide constructs not bound to the first or the second target molecule is less than a z-score of at least one peptide construct of the first library of peptide constructs bound to the first or second target molecule. The z-score of a peptide is calculated by first determining a relative abundance level of each peptide constructs in the library of peptide constructs and then grouping the grouping peptide constructs into bins based on similarity of relative abundance level, wherein each bin comprises at least 300 peptide constructs. The relative abundance level of each peptide construct is also normalized against the average of the relative abundance level of the negative control peptide constructs in the library of peptide constructs. The normalized relative abundance levels of each peptide construct in a bin are used to determine a mean and a standard deviation of each bin. The z-score of a peptide is calculated based on the mean and a standard deviation of its bin. In some aspects, the determination of the mean and the standard deviation of the normalized relative abundance levels in a bin excludes peptide constructs having outlier relative abundance levels. In some aspects, a peptide construct has an outlier relative abundance level when its normalized relative abundance level is outside the 95% highest density interval of its bin. In certain implementations, 5% of peptide constructs in each bin are excluded from the determination of the mean and the standard deviation of the normalized relative abundance levels in a bin.

The methods further comprise identifying a first peptide from the at least one peptide construct of the first library of peptide constructs bound the first target molecule. The z-score of the first peptide from the first binding assay is a threshold z-score and the z-score of the first peptide in the second binding assay is less than the threshold z-score. Next, the methods comprise generating a second library of peptide constructs based on the first peptide to identify a peptide with differential specific binding to the first and the second target molecules and identifying at least one variant peptide from the second library of peptide constructs with increased binding to the first target molecule compared to that of the first peptide and decreased binding to the second target molecule compared to that of the first peptide. Increased binding is indicated by a higher z-score than the identified threshold z-score of the first peptide. In some aspects, the methods comprise separating the at least one peptide construct of the first library of peptide constructs bound to the first target molecule from the at least one peptide constructs of the first library of peptide constructs not bound to the first target molecule.

The second library of peptide constructs comprises a peptide construct comprising the first peptide and a plurality of peptide constructs comprising variant peptides. In some aspects, the variant peptides of the plurality of peptide constructs are produced by complete single residue mutagensis. Such variant peptide differs from the first peptide by a single point mutation, which is a substitution of the original amino acid with the nineteen other amino acids. Thus, the plurality of peptide constructs comprises nineteen different variants peptides for each substituted residue of the first peptide. In other aspects, the variant peptides of the plurality of peptide constructs are created by sliding window mutagenesis. Thus, each variant peptide differs from the first peptide by at least two contiguous residues from either the C-terminus end or the N-terminus end of the first peptide. In still other aspects, the variant peptides of the plurality of peptide constructs are produced by alanine scanning mutagenesis. In one aspect, these variant peptides differ from the first peptide by a single point mutation and each point mutation is a substitution with alanine. In another aspect, each point mutation is a substitution with glycine. In certain implementations, the plurality of variant peptides comprises at least one of the sets of variant peptides produce by complete single residue mutagenesis, sliding window mutagenesis, and alanine scanning mutagenesis. In some aspects, the first peptide comprises a consensus sequence generated from bound peptides. Accordingly, in some embodiments, the plurality of peptide constructs comprises variant constructs comprising a core sequence having at least 5 consecutive amino acids from the consensus sequence. In particular embodiments, at least two variant peptides are identified from the library of peptide constructs with increased binding to the target molecule compared to that of the first peptide. Such methods further comprise generating a second library of peptide constructs that comprises multimers of the at least two variant peptides. In some aspects, the multimers are dimers, for example a heterodimer formed by two variant peptides identified to have increased binding to the target molecule compared to that of the first peptide.

In some embodiments of the method of identifying a peptide with differential specific binding to the first target molecule and a second target molecule, the first target molecule is a tumor cell, the second target molecule is a normal cell having the same histologic type as the tumor cell, and the at least one peptide construct with differential specific binding recognizes the tumor cell with higher affinity than the normal cell. For example, the first target molecule is a mutant signaling cascade enzyme from a tumor cell, the second target molecule is a corresponding wild-type signaling cascade enzyme from a normal cell having the same histologic type as the tumor cell; and the at least one peptide construct with differential specific binding recognizes the mutant signaling cascade enzyme with higher affinity than the wild-type signaling cascade enzyme. In particular embodiments, the mutant signaling cascade enzyme and the wild-type signaling cascade enzyme are protein kinases.

In some aspects of the methods of identifying a peptide with increased specific binding to a target molecule and the methods of identifying a peptide with increased specific binding to a first target molecule and with differential specific binding to the first target molecule and a second target molecule, each of the peptide constructs of the first library of peptide constructs comprises a peptide portion comprising the first peptide or the variant peptide and an identifying nucleic acid portion that identifies the peptide portion. In some aspects, the identifying nucleic acid portion comprises a polynucleotide sequence or complement thereof encoding the peptide portion. The binding interaction between the peptide constructs with the target molecule is between its peptide portion and the target molecule. In some aspects, the identifying nucleic acid portion of each peptide construct encodes at least 5 randomized amino acids, and the identifying nucleic acid portions are generated with full nucleotide randomization at the first and second positions of each of at least 5 randomized codons and G/T randomization at the third position to minimize stop codons and maximize synthetic yield.

In certain implementations, the step of separating the at least one peptide construct of the first library of peptide constructs bound the target molecule from the at least one peptide constructs of the first library of peptide constructs not bound to the target molecule further comprises immobilization and/or precipitation of the at least one peptide construct capable of specific binding to the target molecule using a capture agent having specific binding to the target molecule. For example, immunoprecipitation with an antibody or antigen-binding fragment having specific binding to the target molecule is used to separate the at least one peptide construct of the first library of peptide constructs bound the target molecule. The separating step may also comprise separating the peptide constructs based on differences in size after contacting the target molecule with the first library, for example, via filtration, centrifugation, size exclusion chromatography, or combinations thereof.

For embodiments of the methods where each of the peptide constructs of the first library of peptide constructs comprises a peptide portion and an identifying nucleic acid portion that identifies the peptide portion, the methods may further comprise sequencing all or a portion of the identifying nucleic acid portion of the at least one peptide construct of the first library of peptide constructs bound to the target molecule. For example, the sequencing step comprises amplification and next generation sequencing of the identifying nucleic acid portion. In some aspects of such embodiments, the method further comprises immobilizing the peptide portion of the variant construct with increased specific binding to the target molecule or with differential specific binding to the first and the second target molecules to a platform matrix or membrane to produce a diagnostic assay or detection kit. The peptide portion of the variant construct may be immobilized with an affinity tag/recognition entity interaction. For example, polyhistidine/NTA/Ni²⁺, glutathione S-transferase/glutathione, maltose binding protein/maltose, streptavidin/biotin, biotin/streptavidin, or antigen (or antigen fragment)/antibody (or antibody fragment). In certain implementations, the diagnostic assay or detection kit is a lateral flow assay.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 depicts a step-wise workflow for efficient synthesis and analysis of large libraries of DNA-barcoded peptides. The peptide-DNA library depicted shows a puromycin (“Puro”) adaptor that facilitates linking of the DNA to the encoded peptide.

FIG. 2 depicts the basic structure of an individual peptide-DNA conjugate.

FIG. 3 depicts the results of a screen where a peptide-DNA conjugate library of 30,069 19-amino acid long peptides from 190 genes in Burkholderia pseudomallei along with 500 random control peptides were incubated with the anti-GroEL monoclonal antibody 8E4 to identify peptides expressing epitopes recognized by the antibody.

FIG. 4 depicts the signal to noise ratio for the 14 hits identified in the screen with the B. pseudomallei peptide-DNA conjugate library and the anti-GroEL monoclonal antibody 8E4.

FIG. 5 depicts the low background reactivity in the screen with the B. pseudomallei PepSeq library and the anti-GroEL monoclonal antibody 8E4.

FIG. 6A depicts the binding of the GroEL monoclonal antibodies, 8E4, 18E7, and 7D10, to 19-amino-acid-long peptides generated from the amino acid sequence of GroEL-1 (locus tag BPSL2697) or of GroEL-2 (locus tag BPSS0477). The upper figure depicts binding with both GroEL-1 and GroEL-2 while the lower figure shows only binding with GroEL-1 and the K_{d} values determined for each antibody. The peptides were identified with a screen using a peptide-DNA conjugate library of about 30,000 unique peptides tiled across 180 B. pseudomallei proteins. FIG. 6B depicts the linear relationship between the K_{d} of a binder (e.g., the monoclonal antibody 8E4) and the number of ligands bound. FIGS. 6C, 6D, and 6E demonstrate alignment of the identified peptides that bound with each monoclonal antibody and the identification of amino acid sequences producing epitopes recognized by each antibody.

FIG. 7 depicts an outline of a method to use a peptide-DNA conjugate library to discover binders for a particular target protein recognized by a monoclonal antibody.

FIG. 8 depicts a pipeline for probe discovery and validation beginning with binder discovery and ending with incorporation of the binder into an assay such as a lateral flow assay (LFA).

FIG. 9 depicts an example of a timeline for binder discovery and affinity maturation.

FIG. 10 depicts the components of a lateral flow assay.

FIG. 11 depicts a configuration of a capture peptide and a reporter peptide in a lateral flow assay.

FIGS. 12A and 12B depict an exemplary schematic of directed library design plan to binder confirmation and maturation. FIG. 12A depicts a complete single amino acid mutagenesis. The original peptide sequence is changed to one of the 19 other amino acid possibilities. This would occur at 30 positions (570 total peptides). FIG. 12B depicts a sliding window mutagenesis where either the C-terminus or the N-terminus is removed and replaced with random additions to generate 30-residue-long peptides.

FIGS. 13A-13B depict binding analysis of the original Burk_A_13002 peptide and amino acid substituted peptides to the 8E4 mAb. FIG. 13A depicts the original GroEL peptide binding. The Y axis shows the z-score for each peptide comparison and each dot represents a different comparison. There were 12 comparisons for 8E4 interaction at room temperature wash (black) compared to other target comparisons (i.e. 8E4 warm wash vs 7D10 cold wash, etc.). The grey circles are the same peptide bound to other targets and compared to the same bins (i.e. 7D10 warm wash vs 8E4 warm wash, etc.). The grey line represents the average z-score for the 8E4 bound original peptide and is carried through FIG. 13A and FIG. 13B as a comparison. FIG. 13B depicts the substitutional mutagenesis of the original GroEL peptide at each amino acid position. The Y axis shows the z-score for each peptide comparison and each dot represents the average z-score signal for a different peptide. The peptides in each column represent a change that position from the original peptide sequence (19 possible amino acid changes per location) shown on the bottom. The substituted amino acids are color coded to represent the different classes of amino acids. The original require epitope is underlined.

FIGS. 14A-14B depict the binding of original PV1_079508 peptide and amino acid substituted peptides to the virB5 target. FIG. 14A depicts the original PV1 peptide binding. The Y axis shows the z-score for each peptide comparison and each dot represents a different comparison. There were 20 comparisons for virB5 interaction (black) compared to other target comparisons (i.e. virB5 vs trwG, etc.). The grey circles are the same peptide bound to other targets and compared to the same bins (i.e trwG vs virB5 or E1/E2 vs trwG, etc.). The gray line represents the average Z-score for the virB5 bound original peptide (black dots) and is carried through FIG. 14A and FIG. 14B as a comparison. FIG. 14B depicts the substitutional mutagenesis of the original peptide at each amino acid position. The Y axis shows the z-score for each peptide comparison and each dot represents the average z-score signal for a different peptide. The peptides in each column represent a change at that position (19 possible amino acid changes per location) from the original peptide sequence shown on the bottom. The substituted amino acids are color coded to represent the different classes of amino acids. The original require epitope is underlined.

FIGS. 15A-15C depict epitope-resolved CoV serology using a highly-multiplexed peptide-based assay based on peptide-DNA conjugates. FIG. 15A depicts peptide coverage depth across the SARS-CoV-2 spike (S) and nucleocapsid (N) proteins within the ‘SCV2’ peptide library. Peptide coverage depth (top line; blue) correlates well with amino acid sequence diversity within the target SARS-CoV-2 sequences (bottom line; green), calculated as the number of unique 30 mers. FIG. 15B shows the number of peptides within the HV library that were designed from each of the six human CoVs known prior to 2019. FIG. 15C depicts an example scatter plot illustrating SCV2 peptide-DNA conjugate assay results for a single serum sample. This plot shows normalized sequence read counts (log 10 scale) for each peptide in the SCV2 library. Assay results using antibody-free negative controls are shown on the x-axis (average of 8 replicates shown), while the results from a COVID-19 convalescent serum sample are shown on the y-axis (average of 2 replicates shown). Grey circles represent unenriched peptides, with a strong correlation between the two assays, based on the starting abundance of the different peptides. Gray circles with a plus sign represent SARS-CoV-2 and black circles represent non-SARS-CoV-2 control peptides that have been enriched through interaction with serum antibodies.

FIGS. 16A-16E depict how peptide-DNA conjugates identify recurrent reactivities to SARS-CoV-2 peptides and classifies exposure status. FIG. 16A shows boxplots showing the number of enriched SCV2 library peptides from assays with negative control (dark gray, n=68) and COVID-19 convalescent (light gray, n=55) samples, divided into three different categories: non-SARS-CoV-2 control peptides (Control), and SARS-CoV-2 Spike (S) and Nucleocapsid (N) peptides. “***”=t-test with p<1⁻⁵, “NS”=not significant). Individual data points are shown as circles, the limits of the boxes correspond to the 1st and 3rd quartiles, the black line inside each box corresponds to the median and the whiskers extend to points that lie within 1.5 interquartile ranges of the 1st and 3rd quartiles. FIGS. 16B and 16C show heat maps showing the locations of enriched SARS-CoV-2 peptides within the S and N proteins, respectively. Each row represents a single serum/plasma sample, and each plot includes only samples with at least one enriched peptide from the focal protein. Each position is colored according to the number of enriched peptides that overlap that position. The horizontal dashed line separates COVID-19 convalescent samples (top) from negative control samples (bottom). The vertical dashed lines in FIG. 16B represent the S1-S2 and S2′ cleavage sites, respectively. Grey boxes indicate selected functional regions: receptor binding domain (RBD), fusion peptide (FP) and heptad repeat 2 (HR2). FIG. 16D shows boxplots showing the distribution of Z-scores across all assayed samples for the 6 most common epitope reactivities observed in FIG. 16B and 16C. For each sample/epitope combination, the Z-score of the most enriched, overlapping peptide is presented. Boxplots were drawn as described in FIG. 16A with convalescent samples in gray and negative controls in light gray. t-test: “*”=p<0.05, “**”=p<0.01, “NS”=not significant. FIG. 16E illustrates receiver-operating curves showing sensitivity/specificity across a range of thresholds with which logistic regression models trained on randomly selected subsets of 70% of the donors were able to classify the remaining 30% of donors as either negative control or convalescent, using log-transformed Z-scores for the 6 epitopes described in FIG. 16D as features. The red curve shows the average of 100 individual runs. Each patient sample was assayed in duplicate. Enriched peptides were determined based on consistent signal across replicates and Z-scores shown as averages across replicates.

FIGS. 17A-17C depict how SARS-CoV-2 elicits cross-reactive Spike S2 antibodies that preferentially recognize homologs from the endemic CoVs. FIG. 17A shows line plots comparing peptide-specific patterns of enrichment before and after targeted depletion of antibodies binding the SARS-CoV-2 FP or HR2 antigens. Each plot compares peptide enrichment (Z-score) from the same samples prior to (left) and after (right) antibody depletions. Each line represents a single peptide found to be enriched prior to antibody depletion, and the color of each line indicates the species from which the peptide was designed. Each plot includes results from 3-6 convalescent donors. For each depletion experiment (FP- and HR2-targeted), on- and off-target peptides are plotted separately. Dashed horizontal lines represent a Z-score of 8. FIG. 17B shows scatterplots comparing enrichment (Z-score) between SARS-CoV peptides (x-axis) and endemic human CoV peptides (y-axis) across three epitopes (S:FP, S:HR2, and N:166, respectively) and all samples assayed in duplicate using the HV library (average Z-score is shown). Convalescent and negative control samples are represented by orange and blue shapes, respectively. The type of shape indicates the endemic CoV species from which the most highly enriched peptide was designed (circle=HCoV-OC43, square=HCoV-229E, upright triangle=HCoV-HKU1 and upside-down triangle=HCoV-NL63). Shapes with black outlines indicate the samples included in the antibody depletion experiments shown in FIG. 17A. FIG. 17C depicts a ternary plot showing the relative signal across HV library peptides from three human CoVs (SARS-CoV, HCoV-OC43 and HCoV-229E) at three commonly reactive epitopes in COVID-19 convalescent patients. Each point represents a single convalescent sample that exhibited at least one enriched SCV2 library peptide at the relevant epitope. Position within the triangle was determined by normalizing the maximum peptide Z-score (averaged across replicates) observed for each of the three focal species.

### DETAILED DESCRIPTION

As used herein and in the appended claims, the singular forms “a,” “an,” and “the” include plural referents unless the context clearly dictates otherwise. For example, “a” or “an” means “at least one” or “one or more.” Thus, reference to “an antibody or antigen binding fragment thereof” refers to one or more antibodies or antigen binding fragments thereof, and reference to “the method” includes reference to equivalent steps and methods disclosed herein and/or known to those skilled in the art, and so forth.

The terms “target,” “target molecule,” and “target agent” are used interchangeably herein and refer to a protein, toxin, enzyme, pathogen, cell or biomarker that is incubated with a library to identify peptides demonstrating specific binding to the target.

The terms “peptide”, “polypeptide,” and the like are used interchangeably herein, and refer to a polymeric form of amino acids of any length, chemically or biochemically modified or derivatized amino acids, and polypeptides having modified peptide backbones.

The term “peptide construct” as used herein, refers to a peptide of any length with modifications, for example, attached to an identifying oligonucleotide. The attachment of the peptide to the modification may be via an intervening linker and the attachment may be covalent or non-covalent. In some embodiments, the identifying oligonucleotide may be the message that was translated to form the peptide portion of the construct, or it may be any other sequence that is known and can be used to identify the attached peptide by sequencing. ‘Peptide construct sets’ refer to a pool of peptide constructs generated from a custom-designed set of oligonucleotides. The sets may contain as few as one copy per species of peptide construct but typically contain many copies of each peptide construct.

The term “variant peptide” as used herein refers to peptide, for example an original binder, with its amino acid sequence modified, for example, through single residue mutagenesis, sliding window mutagenesis, or alanine scanning mutagenesis. As used herein, the term “original binder” refers to a peptide that has been found to have specific finding to a target molecule.

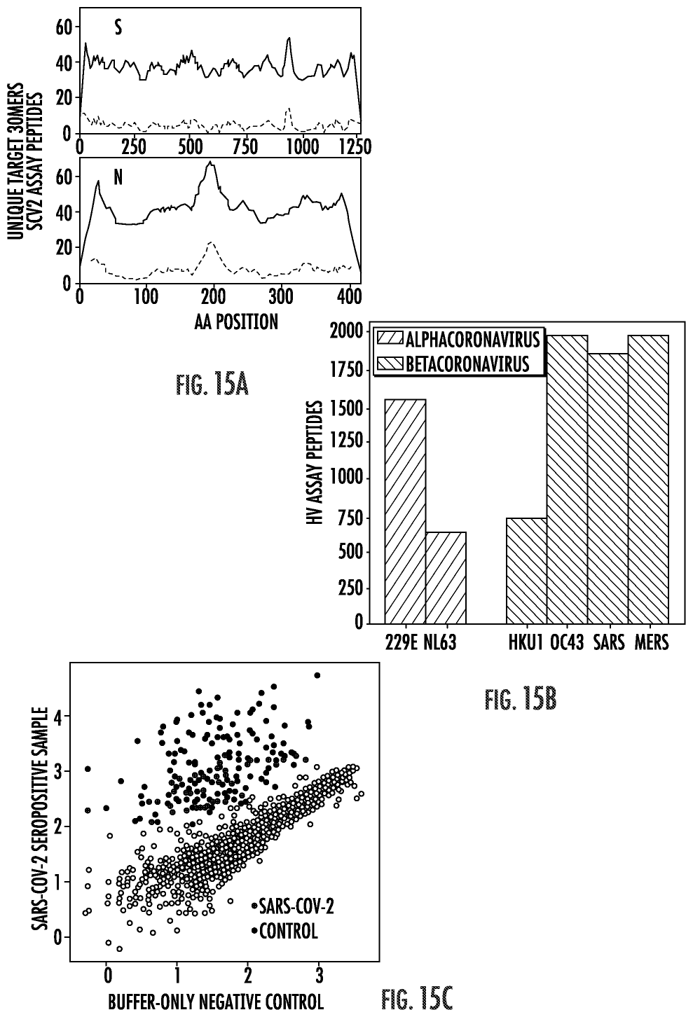

The term “complete single residue mutagenesis” as used herein refers to a method of producing variant peptides where each residue is changed to all of the other possible 19 amino acids (see FIG. 12A). For example, a base peptide of 30 amino acids in length will result in 570 variant peptides (19 amino acids×30 positions). In some aspects, the complete single residue mutagenesis methodology helps to identify which residue or residues would be important for specific binding to the target molecule and how the different chemical properties of amino acids affect the binding affinity.

The term “sliding window” as used herein refers to a method of producing shorter fragments of a full-length peptide by sequentially moving down the amino acid sequence of the full-length peptide by a given number of amino acids (e.g., one, two, three, or more amino acids) to result in replace of continuous amino acid residues from the C-terminus or the N-terminus (see FIG. 12B). The beginning of the sliding window can start at the first amino acid or the last amino acid in the sequence of the full-length peptide or at any subsequent amino acid. The shorter fragments can stretch for at least 5 amino acids, at least 6 amino acids, at least 7 amino acids, at least 8 amino acids, at least 9 amino acids, at least 10 amino acids, at least 15 amino acids, at least 20 amino acids, at least 25 amino acids, or at least 30 amino acids. In some aspects, the sliding window methodology helps to defines specific internal sequences that contribute to binding.

The terms “alanine scan” or “alanine scanning” as used herein refers to a method of producing variant peptides where alanine or glycine is incorporated into the sequence of the original binder. In some aspects, the alanine scan methodology helps to identify which residue or residues would be important for specific binding to the target molecule.

The term “peptidomimetic” as used herein refers to a compound that comprises the same general structure of a corresponding polypeptide, but which includes modifications that increase its stability or biological function. For instance, the peptidomimetic can be a “reverso” analogue of a given peptide, which means that the peptidomimetic comprises the reverse sequence of the peptide. In addition, or instead, the peptidomimetic can comprise one or more amino acids in a “D” configuration (e.g., D-amino acids), providing an “inverso” analogue. Peptidomimetics also include peptoids, wherein the side chain of each amino acid is appended to the nitrogen atom of the amino acid as opposed to the alpha carbon. Peptoids can, thus, be considered as N-substituted glycines which have repeating units of the general structure of NRCH₂CO and which have the same or substantially the same amino acid sequence as the corresponding polypeptide.

The peptides and peptidomimetics described herein can comprise synthetic, non-naturally occurring amino acids. Such synthetic amino acids include, for example, aminocyclohexane carboxylic acid, norleucine, α-amino n-decanoic acid, homoserine, S-acetylaminomethyl-cysteine, trans-3- and trans-4-hydroxyproline, 4-aminophenylalanine, 4-nitrophenylalanine, 4-chlorophenylalanine, 4-carboxyphenylalanine, β-phenylserine β-hydroxyphenylalanine, phenylglycine, α-naphthylalanine, cyclohexylalanine, cyclohexylglycine, indoline-2-carboxylic acid, 1,2,3,4-tetrahydroisoquinoline-3-carboxylic acid, aminomalonic acid, aminomalonic acid monoamide, N′-benzyl-N′-methyl-lysine, N′,N′-dibenzyl-lysine, 6-hydroxylysine, ornithine, α-aminocyclopentane carboxylic acid, α-aminocyclohexane carboxylic acid, α-aminocycloheptane carboxylic acid, α-(2-amino-2-norbornane)-carboxylic acid, α,γ-diaminobutyric acid, α,β-diaminopropionic acid, homophenylalanine, α-tert-butylglycine, and 2-(4-pentenyl)-alanine. The properties of such synthetic amino acids are well-documented. Any natural amino acid of one or more of the sequences discussed herein can be substituted with a synthetic amino acid having similar properties.

As used herein, the term “binding” refers to an attractive interaction between two molecules which results in a stable association in which the molecules are in close proximity to each other. Molecular binding can be classified into the following types: non-covalent, reversible covalent and irreversible covalent. Molecules that can participate in molecular binding include proteins, nucleic acids, carbohydrates, lipids, and small organic molecules such as pharmaceutical compounds. For example, proteins that form stable complexes with other molecules are often referred to as receptors while their binding partners are called ligands. Nucleic acids can also form stable complex with themselves or others, for example, DNA-protein complex, DNA-DNA complex, DNA-RNA complex.

As used herein, the term “specific binding” refers to the specificity of a binder, e.g., an antibody, such that it preferentially binds to a target, such as a polypeptide antigen. When referring to a binding partner, e.g., protein, nucleic acid, antibody or other affinity capture agent, etc., “specific binding” can include a binding reaction of two or more binding partners with high affinity and/or complementarity to ensure selective hybridization under designated assay conditions. Typically, specific binding will be at least three times the standard deviation of the background signal. Thus, under designated conditions the binding partner binds to its particular target molecule and does not bind in a significant amount to other molecules present in the sample. Recognition by a binder or an antibody of a particular target in the presence of other potential interfering substances is one characteristic of such binding. Preferably, binders, antibodies or antibody fragments that are specific for or bind specifically to a target bind to the target with higher affinity than binding to other non-target substances. Also preferably, binders, antibodies or antibody fragments that are specific for or bind specifically to a target avoid binding to a significant percentage of non-target substances present in a testing sample. In some embodiments, binders, antibodies or antibody fragments of the present disclosure avoid binding greater than about 90% of non-target substances, although higher percentages are clearly contemplated and preferred. For example, binders, antibodies or antibody fragments of the present disclosure avoid binding about 91%, about 92%, about 93%, about 94%, about 95%, about 96%, about 97%, about 98%, about 99%, and about 99% or more of non-target substances. In other embodiments, binders, antibodies or antibody fragments of the present disclosure avoid binding greater than about 10%, 20%, 30%, 40%, 50%, 60%, or 70%, or greater than about 75%, or greater than about 80%, or greater than about 85% of non- target substances.

The terms “z-score,” “Z-score,” “Zscore”, and “Z score” are used interchangeably herein and refer to the number of standard deviations away from the mean, with the mean and standard deviation calculated independently for the peptides from each bin. As used herein, the z-score is calculated from a bin-based approach that compares the relative abundance estimates of groups of peptides known to be present at similar starting frequencies in a peptide construct library, which is used to assess binding affinities. For example, a peptide is determined to be bound to the target molecule or not bound to the target molecule in a binding assay by the difference in their measured relative abundance in the bound assay including the target versus the relative abundance in the starting library, as inferred from binding assays that do not include the target. Peptides are assigned to bins based on their relative frequency estimates in multiple negative control assays. Each bin contains at least 300 peptides with similar average relative frequency estimates (relative abundance level) across the negative controls. Peptides within a bin are inferred to be present at similar relative abundances in the starting peptide construct library. In order to account for slight differences in starting abundance between peptides contained in the same bin, relative abundance in experimental samples is first normalized to the corresponding value for the negative controls. These normalized abundances are then used to calculate a z-score for each peptide within each sample. It is important that the mean and standard deviation reflect the distribution of unenriched unbound peptides within a bin. Accordingly, peptides with outlier relative abundance level are excluded from the mean and standard deviation calculations.

The terms “capture agent” and “capture group” as used herein refer to any moiety that allows capture of a target molecule or a peptide construct via binding to or linkage with an affinity group or domain on the target molecule or an affinity tag of the peptide construct. The binding between the capture agent and its affinity tag may be a covalent bond and/or a non-covalent bond. A capture agent includes, e.g., a member of a binding pair that selectively binds to an affinity tag on a fusion peptide, a chemical linkage that is added by recombinant technology or other mechanisms, co-factors for enzymes and the like. Capture agents can be associated with a peptide construct using conventional techniques including hybridization, cross-linking (e.g., covalent immobilization using a furocoumarin such as psoralen), ligation, attachment via chemically-reactive groups, introduction through post-translational modification and the like.

“Sequence determination,” “sequencing,” and the like include determination of information relating to the nucleotide base sequence of a nucleic acid. Such information may include the identification or determination of partial as well as full sequence information of the nucleic acid. Sequence information may be determined with varying degrees of statistical reliability or confidence. In one aspect, the term includes the determination of the identity and ordering of a plurality of contiguous nucleotides in a nucleic acid. “High throughput sequencing” or “next generation sequencing” includes sequence determination using methods that determine many (typically thousands to billions) of nucleic acid sequences in an intrinsically parallel manner, i.e., where DNA templates are prepared for sequencing not one at a time, but in a bulk process, and where many sequences are read out preferably in parallel, or alternatively using an ultra-high throughput serial process that itself may be parallelized. Such methods include but are not limited to pyrosequencing (for example, as commercialized by 454 Life Sciences, Inc., Branford, Conn.); sequencing by ligation (for example, as commercialized in the SOLiD™ technology, Life Technologies, Inc., Carlsbad, Calif.); sequencing by synthesis using modified nucleotides (such as commercialized in TruSeq™ and HiSeq™ technology by Illumina, Inc., San Diego, Calif.; HeliScope™ by Helicos Biosciences Corporation, Cambridge, Mass.; and PacBio RS by Pacific Biosciences of California, Inc., Menlo Park, Calif.), sequencing by ion detection technologies (such as Ion Torrent™ technology, Life Technologies, Carlsbad, Calif.); sequencing of DNA nanoballs (Complete Genomics, Inc., Mountain View, Calif.); nanopore -based sequencing technologies (for example, as developed by Oxford Nanopore Technologies, LTD, Oxford, UK), and like highly parallelized sequencing methods. “Small molecule,” as used herein, means a molecule less than 5 kilodaltons, more typically, less than 1 kilodalton. As used herein, “small molecule” includes peptides.

“Affinity tag” is given its ordinary meaning in the art. An affinity tag is any biological or chemical material that can readily be attached to a target biological or chemical material. Affinity tags may be attached to a target biological or chemical molecule by any suitable method. For example, in some embodiments, the affinity tag may be attached to the target molecule using genetic methods. For example, the nucleic acid sequence coding the affinity tag may be inserted near a sequence that encodes a biological molecule; the sequence may be positioned anywhere within the nucleic acid that enables the affinity tag to be expressed with the biological molecule, for example, within, adjacent to, or nearby. In other embodiments, the affinity tag may also be attached to the target biological or chemical molecule after the molecule has been produced (e.g., expressed or synthesized). As one example, an affinity tag such as biotin may be chemically coupled, for instance covalently, to a target protein or peptide to facilitate the binding of the target to streptavidin.

Affinity tags include, for example, metal binding tags such as histidine tags, GST (in glutathione/GST binding), streptavidin (in biotin/streptavidin binding). Other affinity tags include Myc or Max in a Myc/Max pair, or polyamino acids, such as polyhistidines. At various locations herein, specific affinity tags are described in connection with binding interactions. The molecule that the affinity tag interacts with (i.e., binds to), which may be a known biological or chemical binding partner, is the “recognition entity.” It is to be understood that the invention involves, in any embodiment employing an affinity tag, a series of individual embodiments each involving selection of any of the affinity tags described herein.

A “recognition entity” may be any chemical or biological material that is able to bind to an affinity tag. A recognition entity may be, for example, a small molecule such as maltose (which binds to MBP, or maltose binding protein), glutathione, NTA/Ni²⁺, biotin (which may bind to streptavidin), or an antibody. An affinity tag/recognition entity interaction may facilitate attachment of the target molecule, for example, to another biological or chemical material, or to a substrate (e.g., a nitrocellulose membrane or other immobilized substrate). Examples of affinity tag/recognition entity interactions include polyhistidine/NTA/Ni²⁺, glutathione S-transferase/glutathione, maltose binding protein/maltose, streptavidin/biotin, biotin/streptavidin, antigen (or antigen fragment)/antibody (or antibody fragment), and the like.

The disclosure relates to a screening platform that allows the computational design of diverse molecular libraries and the high capacity screening of them. The binding maturation cycles that follow identify superior binding agents in a directed design, rapid and economical strategy. Methods for identifying synthetic molecular binding agents (SYMBA) from peptide libraries are disclosed. In one aspect, the disclosure is directed to the methods of maturing a peptide library to improve binding to a target molecule. In another aspect, the disclosure is directed to methods of maturing a peptide library to improve binding to a target molecule and methods of methods of identifying a peptide with increased specific binding to a first target molecule and with differential specific binding to the first target molecule and a second target molecule.

The peptide libraries described herein comprise at least 300 peptide constructs, for example, at least 500 peptide constructs or at least 1,000 peptide constructs. Preferably, the peptide libraries comprise at least 100,000 peptide constructs or at least 200,000 peptide constructs. In particular embodiments, the peptide libraries comprise greater than one billion peptide constructs, for example the starting peptide library of the methods. In certain aspects, the use of simple random amino acids libraries is complemented through the design of randomized amino acids in the context of structural motifs.

The target molecule may be a protein, toxin, enzyme, pathogen, cell or biomarker. In some embodiments, the target molecule may be tumor cell. In one aspect, the target molecule is a cell surface protein, a cell surface carbohydrate, or a protein secreted by a cell (for example, a tumor cell). In another aspect, the target molecule is a signaling cascade enzyme. In some embodiments, the signaling cascade enzyme is a protein kinase. In one embodiment, the protein kinase is selected from the group consisting of anaplastic lymphoma kinase (ALK), BCR-Abl tyrosine kinase, serine/threonine-protein kinase B-Raf, bruton agammaglobulinemia tyrosine kinase (BTK), cyclin-dependent kinase (CDK), tyrosine-protein kinase Met (c-Met), epidermal growth factor receptor (EGFR), Janus kinase (JAK), MAPK/ERK kinase (MEK), platelet-derived growth factor receptor (PDGFR), RET tyrosine kinase, tyrosine-protein kinase Src, and vascular endothelial growth factor receptor (VEGFR). In some embodiments, the target molecule is a cell surface protein, a cell surface carbohydrate, or a protein secreted by a pathogen.

In certain embodiments, the target molecule is a tumor cell. Tumor cells can be obtained from a spontaneous tumor which has arisen, e.g., in a human subject or they may be obtained from an experimentally derived or induced tumor, in an animal subject. The tumor cells can be an established tumor cell line having an identical tissue type as the tumor of said tumor-bearing subject. It need not be HLA class II matched to said subject. Further, the tumor cells can be obtained, for example, from a solid tumor of an organ, such as a tumor of the lung, liver, breast, colon, bone, etc. The tumor cells can also be obtained from a blood-borne (i.e., dispersed) malignancy, such as a lymphoma, a myeloma or a leukemia. Tumor cells can also be obtained from a subject by, for example, surgical removal of tumor cells, e.g., a biopsy of the tumor, or from a blood sample from the subject in cases of blood-borne malignancies. In the case of an experimentally induced tumor, the tumor cells used to induce the tumor can be used, e.g., cells of a tumor cell line. The tumor cells include but are not limited to those derived from carcinomas, sarcomas, lymphoma, glioma, melanoma, neuroblastoma and the like. In another embodiment, where differential binding is explore, another target molecule is a normal cell of the same histologic type as a tumor cell. The normal cell can be syngeneic, allogeneic or xenogeneic to the host. The peptide-DNA conjugate platform is well suited for producting the peptide libraries described herein. According to various embodiments, a peptide-DNA conjugate method comprises a method for pooled, highly-parallel expression of proteins, each associated with a nucleic acid barcode. In some embodiments, the peptide-DNA conjugate method comprises a method for pooled, highly-parallel in vitro expression of proteins, each covalently linked to a DNA barcode through a puromycin-containing linkage. In other embodiments, the peptide-DNA conjugate method comprises phage display, mRNA display, or other method. The peptide-DNA conjugate platform allows for rapidly screening of large populations of diverse molecules for candidate SYMBAs and further exploration of the chemical structural space of these candidates for higher affinity and more specific binding agents. In some embodiments, the present invention provides the following peptide-DNA-4-SYMBA strategy.
1. The peptide-DNA conjugate approach is a proprietary technology that can rapidly generate a large number of potential synthetic binding agents. The peptide-DNA conjugate approach is a method that generates diverse peptide libraries (10-50 amino acids long) with each peptide conjugated to a unique DNA tag that can be used to monitor peptide abundance following binding experiments. It has been successfully used to explore epitope complexity in humoral immunological responses to disease and MHC class II binding of epitopes. Importantly, the peptide sequences can be used in large multiplexed binding assays with upwards of 100,000 of unique programmable peptides (e.g., from predicted coding regions in pathogen genomes), or le10 randomized peptides.
2. Binding assays for any molecular target can be used to screen large diverse peptide-DNA conjugate libraries. A biological structure or molecule can be mixed with diverse peptides, separated from unbound peptides, and then queried for those that “stick.” For example, a viral coat protein or toxin can be screened for binding to upwards of 10 billion individual peptides in a single binding assay. The particular libraries could be constrained “random” libraries or those intelligently designed libraries based upon prior knowledge of the target.
3. Candidate affinity agents can be used to guide the search for higher and better agents in the same chemical space. For example, a low affinity peptide binder can be extended and/or altered at a single or multiple amino acid positions to create a focused peptide-DNA conjugate library in the same “chemical space”. This approach is rapid and high-throughput, as the alterations can be made in silico followed by commercial oligonucleotide synthesis, and it can be done once or multiple times to find the best SYMBA (i.e., higher affinity, sensitivity and specificity). It is a focused hierarchical process that can examine le5-1e10 peptides in each cycle to hone in on the best agent possible.
4. Molecular diversity of the libraries can be generated through the generation of random amino acid sequences, but it can also be constrained to increase the frequency of high affinity binding agents. Particular repertoires of amino acids (acids, bases, and hydrophobic) are more likely to bind a target, while others are less likely (e.g., glycine). In addition, more conformed structures can be made—e.g., cyclic polypeptides through the use of disulfide cysteine bridges, or even small folded domains comprising a backbone of constant sequence that brings together variable target-binding polypeptide loops. Finally, modified amino acids can be introduced to increase the potential diversity beyond the standard 20 biological amino acids.
5. SYMBAs can be used in many different detector/diagnostic devices. The chemistry to attach short peptides to a matrix or a fluorescent reporter is well developed.

The peptide-DNA conjugate platform is described in greater detail in U.S. Pat. Nos. 9,958,454; 10,288,608; and U.S. Patent Application Publication No. 2016/0025726, the contents of which are hereby incorporated by reference.

In some aspects, the disclosed methods comprise sequential binding assays with an enrichment step between each to increase the binding signal relative to the binding noise in the assay. This is accomplished by an initial binding assay that targets rare binders in very complex peptide libraries. For example, individual peptides will have relatively low abundance in complex peptide libraries with greater than a billion peptides. Identification of these strong but rare binders can be enhanced through an enrichment step followed by a subsequent repeat of the binding assay. For example, with a peptide-DNA conjugate library, this involves an initial binding assay, the PCR amplification of the DNA tag portions of the bound peptide-DNA components, the resynthesis of a peptide-DNA conjugate library from PCR amplicons, and then the rebinding of this enriched library. In other systems, such as phage display, this enrichment procedure has been called “panning.”

In some embodiments, the disclosed methods are used for drug discovery. Once peptides are found that bind to biological targets, there is the potential that they have potential for use as drugs. The binding of small molecules is the basis of therapeutic action by many drugs and peptide-DNA conjugate libraries represents a rapid approach for identifying candidates. This is particularly true if the binding assays include biological components that are involved in key pathways for disease progression. This is true for infectious diseases but also physiological and oncological diseases. In practice, this involves peptide-DNA conjugate library binding to key biological components, the identification of high affinity ligands, and the in vivo testing of the binders in a disease model. Alteration of the disease progression through the addition of the high affinity binders is evidence of efficacy.

In other embodiments, the disclosed methods are used for identifying that variations in an epitope that would still enable the binding to the antibody against the epitope, which can predict how variant strains of a pathogen may be protected by a vaccination strategy or treated by an antibody therapy.

In some embodiments, the disclosed methods further comprise evaluating the peptide portion of the at least one variant peptide construct capable of increased specific binding to the target molecule for biological activity in cell culture to develop a therapeutic agent.

In other embodiments, the disclosed methods further comprise compiling a map of peptide constructs capable of specific binding to the target molecule to identify at least one binding site on the target molecule for targeting with a therapeutic agent.

In one embodiment, the therapeutic agent is a peptide or peptidomimetic comprising an amino acid sequence from a peptide portion of a peptide construct with high affinity to the target molecule or the inverse thereof. In certain aspects, the amino acid sequence comprises at least 5 consecutive amino acids from the amino acid sequence from a peptide portion of a peptide construct with high affinity to the target molecule or the inverse thereof. In other aspects, the amino acid sequence comprises at least 6 consecutive amino acids, at least 7 consecutive amino acids, at least 8 consecutive amino acids, at least 9 consecutive amino acids, at least 10 consecutive amino acids, at least 15 consecutive amino acids, or at least 20 consecutive amino acids from the amino acid sequence from a peptide portion of a peptide construct with high affinity to the target molecule or the inverse thereof.

In one embodiment, the therapeutic agent is a protein kinase inhibitor. In another embodiment, the target molecule is an enzyme and activity of the enzyme is determined with and without the therapeutic agent to confirm the efficacy of the therapeutic agent.

### Methods of Maturing a Peptide Library

The methods of maturing a peptide library to improve binding to a target molecule comprise identifying a first peptide having specific binding to the target molecule has and having an identified a threshold z-score and then generating a library of peptide constructs based on the first peptide. The library of peptide constructs comprises the first peptide and a plurality of variant peptides. The method further comprises contacting the target molecule with the library of peptide constructs to perform a second binding assay and identifying at least one variant peptide from the second binding assay with increased binding to the target molecule compared to that of the first peptide. Increased binding is indicated by a z-score higher than the threshold z-score.

In some aspects, the variant peptides of the plurality of peptide constructs are produced by complete single residue mutagensis. Such variant peptide differs from the first peptide by a single point mutation, which is a substitution of the original amino acid with the nineteen other amino acids. Thus, the plurality of peptide constructs comprises nineteen different variants peptides for each substituted residue of the first peptide. In other aspects, the variant peptides of the plurality of peptide constructs are created by sliding window mutagenesis. Thus, each variant peptide differs from the first peptide by at least two contiguous residues from either the C-terminus end or the N-terminus end of the first peptide. In still other aspects, the variant peptides of the plurality of peptide constructs are produced by alanine scanning mutagenesis. In one aspect, these variant peptides differ from the first peptide by a single point mutation and each point mutation is a substitution with alanine. In another aspect, each point mutation is a substitution with glycine. In certain implementations, the plurality of variant peptides comprises at least one of the sets of variant peptides produce by complete single residue mutagenesis, sliding window mutagenesis, and alanine scanning mutagenesis. In some aspects, the first peptide comprises a consensus sequence generated from bound peptides. Accordingly, in some embodiments, the plurality of peptide constructs comprises variant constructs comprising a core sequence having at least 5 consecutive amino acids from the consensus sequence. In some aspects, the core sequence comprises at least 6 consecutive amino acids, at least 7 consecutive amino acids, at least 8 consecutive amino acids, at least 9 consecutive amino acids, at least 10 consecutive amino acids, at least 15 consecutive amino acids, or at least 20 consecutive amino acids from the consensus sequence.

In particular embodiments, at least two variant peptides are identified from the library of peptide constructs with increased binding to the target molecule compared to that of the first peptide. In such implementations, the threshold z-score is the highest z-score of the selected peptides. Such methods further comprise generating a second library of peptide constructs that comprises multimers of the at least two variant peptides. In some aspects, the multimers are dimers, for example a heterodimer formed by two variant peptides identified to have increased binding to the target molecule compared to that of the first peptide. In some embodiments, the dimers comprise a linker between the peptide constructs. In certain embodiments, the linker is selected from the group consisting of a linker with the repeated motif of (GGGS)ₙ, a linker with the repeated motif of (GGGGS)ₙ, a linker with repeated glycines only, a linker with the repeated motif of (EAAAK)ₙ, a poly(ethylene glycol) or PEG-linker, and combinations thereof.

In some embodiments, the methods of maturing a peptide library to improve binding to a target molecule comprise further comprises generating a second library of peptide constructs. The second library of peptide constructs is based on a second peptide that is identified from the second binding assay as having increased binding to the target molecule compared to that of the first peptide. Thus, the second library of peptide constructs comprises a peptide construct comprising the second peptide and a second plurality of peptide constructs comprising variant peptides. In some implementations, the second plurality of peptide constructs comprises variant peptides produced by alanine scanning mutagenesis. Accordingly, the variant peptides differ from the second peptide by a single point mutation and each point mutation is a substitution with alanine or glycine. Such methods further comprise contacting the target molecule with the second library of peptide constructs and identifying at least one variant peptide from the second library of peptide constructs with increased binding to the target molecule compared to that of the second peptide. The at least one variant peptide from the second library of peptide constructs with increased binding to the target molecule compared to that of the second peptide has a higher z-score than the z-score of the second peptide.

Methods of Identifying a Peptide with Increased Specific Binding to a Target Molecule

The methods of identifying a peptide with increased specific binding to a target molecule comprise providing a first library of peptide constructs and contacting the target molecule with the first library of peptide constructs in a first binding assay to produce at least one peptide construct of the first library of peptide constructs bound to the target molecule and at least one peptide construct of the first library of peptide constructs not bound to the target molecule. The z-score of at least one peptide construct of the first library of peptide constructs not bound to the target molecule is less than a z-score of at least one peptide construct of the first library of peptide constructs bound to the target molecule. The methods of identifying a peptide with increased specific binding to a target molecule further comprise identifying a first peptide from the at least one peptide construct of the first library of peptide constructs bound the target molecule, generating a second library of peptide constructs based on the first pepetide to identify a higher affinity peptide, and identifying at least one variant peptide from the second library of peptide constructs with increased binding to the target molecule compared to that of the first peptide. The z-score of the first peptide from the first binding assay is a threshold z-score. Thus, the z-score of the identified at least one variant peptide with increased binding to the target molecule has a higher z-score than the threshold z-score.

The second library of peptide constructs comprises a peptide construct comprising the first peptide and a plurality of peptide constructs comprising variant peptides. In some aspects, the variant peptides of the plurality of peptide constructs are produced by complete single residue mutagensis. Such variant peptide differs from the first peptide by a single point mutation, which is a substitution of the original amino acid with the nineteen other amino acids. Thus, the plurality of peptide constructs comprises nineteen different variants peptides for each substituted residue of the first peptide. In other aspects, the variant peptides of the plurality of peptide constructs are created by sliding window mutagenesis. Thus, each variant peptide differs from the first peptide by at least two contiguous residues from either the C-terminus end or the N-terminus end of the first peptide. In still other aspects, the variant peptides of the plurality of peptide constructs are produced by alanine scanning mutagenesis. In one aspect, these variant peptides differ from the first peptide by a single point mutation and each point mutation is a substitution with alanine. In another aspect, each point mutation is a substitution with glycine. In certain implementations, the plurality of peptides constructs comprises at least one of sets of variant peptide produce by complete single residue mutagenesis, sliding window mutagenesis, and alanine scanning mutagenesis. The plurality of peptide constructs may also comprise variant peptides that comprises at least five consecutive amino acids from the first peptide and at least one of the five consecutive amino acids in the variant peptide construct is substituted with a different amino acid. In some aspects, the first peptide comprises a consensus sequence generated from bound peptides of the first binding assay. Accordingly, in some embodiments, the plurality of peptide constructs comprises variant constructs comprising a core sequence having at least 5 consecutive amino acids from the consensus sequence. In some aspects, the core sequence comprises at least 6 consecutive amino acids, at least 7 consecutive amino acids, at least 8 consecutive amino acids, at least 9 consecutive amino acids, at least 10 consecutive amino acids, at least 15 consecutive amino acids, or at least 20 consecutive amino acids from the consensus sequence.

In particular embodiments, at least two variant peptides are identified from the first library of peptide constructs with increased binding to the target molecule compared to that of the first peptide. In such implementations, the threshold z-score is the highest z-score of the selected peptides. Such methods further comprise generating a second library of peptide constructs that comprises multimers of the at least two variant peptides. In some aspects, the multimers are dimers, for example a heterodimer formed by two variant peptides identified to have increased binding to the target molecule compared to that of the first peptide. In some embodiments, the dimers comprise a linker between the peptide constructs. In certain embodiments, the linker is selected from the group consisting of a linker with the repeated motif of (GGGS)ₙ, a linker with the repeated motif of (GGGGS)ₙ, a linker with repeated glycines only, a linker with the repeated motif of (EAAAK)ₙ, a poly(ethylene glycol) or PEG-linker, and combinations thereof. For such embodiments, the step of identifying at least one variant peptide from the second library of peptide constructs with increased binding to the target molecule compared to that of the first peptide comprise screening bivalent ligands. Candidate ligands can be combined into a single longer peptide to take advantage of both binding moieties. It has been shown that linking via a tether of two weak binders can results in the generation of a high binder. Peptide-DNA conjugate libraries can be used to screen these combined ligand moieties. In practice, this would involve the screening of complex peptide-DNA conjugate libraries to identify binding moieties. Candidate binding peptides can be combined in different pairwise arrangements in a new peptide-DNA conjugate library, which is subjected to the same binding assay. The pairwise binding moieties are separated by different length spacers to allow for spatial constraints between the binding sites.

In some aspects, each of the peptide constructs of the first library of peptide constructs comprises a peptide portion and an identifying nucleic acid portion that identifies the peptide portion. The at least one peptide construct of the first library of peptide constructs bound to the target molecule is bound at its peptide portion to the target molecule. In some embodiments, the the identifying nucleic acid portion of each peptide construct comprises a polynucleotide sequence or complement thereof encoding the peptide portion of the peptide construct. In some aspects, the identifying nucleic acid portion of each peptide construct encodes at least 5 randomized amino acids, and the identifying nucleic acid portions are generated with full nucleotide randomization at the first and second positions of each of at least 5 randomized codons and G/T randomization at the third position to minimize stop codons and maximize synthetic yield. For embodiments of the methods where each of the peptide constructs of the first library of peptide constructs comprises a peptide portion and an identifying nucleic acid portion that identifies the peptide portion, the methods may further comprise sequencing all or a portion of the identifying nucleic acid portion of the at least one peptide construct of the first library of peptide constructs bound to the target molecule. For example, the sequencing step comprises amplification and next generation sequencing of the identifying nucleic acid portion. In some aspects of such embodiments, the method further comprises immobilizing the peptide portion of the variant construct with increased specific binding to the target molecule or with differential specific binding to the first and the second target molecules to a platform matrix or membrane to produce a diagnostic assay or detection kit. The peptide portion of the variant construct may be immobilized with an affinity tag/recognition entity interaction. For example, polyhistidine/NTA/Ni²⁺, glutathione S-transferase/glutathione, maltose binding protein/maltose, streptavidin/biotin, biotin/streptavidin, or antigen (or antigen fragment)/antibody (or antibody fragment). In certain implementations, the diagnostic assay or detection kit is a lateral flow assay.

In some implementations, the methods comprise separating the at least one peptide construct of the first library of peptide constructs bound the target molecule from the at least one peptide construct of the first library of peptide constructs not bound to the target molecule. For example, the method further comprises immobilization and/or precipitation of the at least one peptide construct capable of specific binding to the target molecule using a capture agent having specific binding to the target molecule. In one implementation, immunoprecipitation with an antibody or antigen-binding fragment having specific binding to the target molecule is used to separate the at least one peptide construct of the first library of peptide constructs bound the target molecule. The separating step may also comprise separating the peptide constructs based on differences in size after contacting the target molecule with the first library, for example, via filtration, centrifugation, size exclusion chromatography, or combinations thereof.

Methods of Identifying a Peptide with Differential Binding to Target Molecules

Also described are methods of identifying a peptide with differential specific binding to a first target molecule and a second target molecule. The methods comprise providing a first library of peptide constructs and contacting the first library of peptide constructs with the first target molecule in at a first binding assay and with the second target molecule in a second binding assay. The first and the second binding assays produce at least one peptide construct of the first library of peptide constructs bound to the first target molecule, at least one peptide construct of the first library of peptide constructs not bound to the target molecule, at least one peptide construct of the first library of peptide constructs bound to the second target molecule, and at least one peptide construct of the first library of peptide constructs not bound to the second target molecule. The z-score of at least one peptide construct of the first library of peptide constructs not bound to the first or the second target molecule is less than a z-score of at least one peptide construct of the first library of peptide constructs bound to the first or second target molecule.

The methods further comprise identifying a first peptide from the at least one peptide construct of the first library of peptide constructs bound the first target molecule. The z-score of the first peptide from the first binding assay is a threshold z-score and the z-score of the first peptide in the second binding assay is less than the threshold z-score. Next, the methods comprise generating a second library of peptide constructs based on the first peptide to identify a peptide with differential specific binding to the first and the second target molecules and identifying at least one variant peptide from the second library of peptide constructs with increased binding to the first target molecule compared to that of the first peptide and decreased binding to the second target molecule compared to that of the first peptide. Increased binding is indicated by a higher z-score than the threshold z-score of the first peptide.

The second library of peptide constructs comprises a peptide construct comprising the first peptide and a plurality of peptide constructs comprising variant peptides. In some aspects, the variant peptides of the plurality of peptide constructs are produced by complete single residue mutagensis. Such variant peptide differs from the first peptide by a single point mutation, which is a substitution of the original amino acid with the nineteen other amino acids. Thus, the plurality of peptide constructs comprises nineteen different variants peptides for each substituted residue of the first peptide. In other aspects, the variant peptides of the plurality of peptide constructs are created by sliding window mutagenesis. Thus, each variant peptide differs from the first peptide by at least two contiguous residues from either the C-terminus end or the N-terminus end of the first peptide. In still other aspects, the variant peptides of the plurality of peptide constructs are produced by alanine scanning mutagenesis. In one aspect, these variant peptides differ from the first peptide by a single point mutation and each point mutation is a substitution with alanine. In another aspect, each point mutation is a substitution with glycine. In certain implementations, the plurality of variant peptides comprises at least one of the sets of variant peptides produce by complete single residue mutagenesis, sliding window mutagenesis, and alanine scanning mutagenesis. In some aspects, the first peptide comprises a consensus sequence generated from bound peptides. Accordingly, in some embodiments, the plurality of peptide constructs comprises variant constructs comprising a core sequence having at least 5 consecutive amino acids from the consensus sequence. In some aspects, the core sequence comprises at least 6 consecutive amino acids, at least 7 consecutive amino acids, at least 8 consecutive amino acids, at least 9 consecutive amino acids, at least 10 consecutive amino acids, at least 15 consecutive amino acids, or at least 20 consecutive amino acids from the consensus sequence. In some implementations, the second library of peptide constructs is re-tested to identify variant peptide constructs with high affinity for the target molecule(s).

In particular embodiments, at least two variant peptides are identified from the library of peptide constructs with increased binding to the target molecule compared to that of the first peptide. In such implementations, the threshold z-score is the highest z-score of the selected peptides. Such methods further comprise generating a second library of peptide constructs that comprises multimers of the at least two variant peptides. In some aspects, the multimers are dimers, for example a heterodimer formed by two variant peptides identified to have increased binding to the target molecule compared to that of the first peptide. In some embodiments, the dimers comprise a linker between the peptide constructs. In certain embodiments, the linker is selected from the group consisting of a linker with the repeated motif of (GGGS)ₙ, a linker with the repeated motif of (GGGGS)ₙ, a linker with repeated glycines only, a linker with the repeated motif of (EAAAK)ₙ, a poly(ethylene glycol) or PEG-linker, and combinations thereof. For such embodiments, the steps of identifying at least one variant peptide from the second library of peptide constructs with increased binding to the first target molecule compared to that of the first peptide and of identifying at least one variant peptide from the second library of peptide constructs with increased binding to the first target molecule compared to that of the first peptide and decreased binding to the second target molecule compared to that of the first peptide comprise screening bivalent ligands. Candidate ligands can be combined into a single longer peptide to take advantage of both binding moieties. It has been shown that linking via a tether of two weak binders can results in the generation of a high binder. Peptide-DNA conjugate libraries can be used to screen these combined ligand moieties. In practice, this would involve the screening of complex peptide-DNA conjugate libraries to identify binding moieties. Candidate binding peptides can be combined in different pairwise arrangements in a new peptide-DNA conjugate library, which is subjected to the same binding assay. The pairwise binding moieties are separated by different length spacers to allow for spatial constraints between the binding sites.

In some embodiments of the method of identifying a peptide with differential specific binding to the first target molecule and a second target molecule, the first target molecule is a tumor cell, the second target molecule is a normal cell having the same histologic type as the tumor cell, and the at least one peptide construct with differential specific binding recognizes the tumor cell with higher affinity than the normal cell. For example, the first target molecule is a mutant signaling cascade enzyme from a tumor cell, the second target molecule is a corresponding wild-type signaling cascade enzyme from a normal cell having the same histologic type as the tumor cell; and the at least one peptide construct with differential specific binding recognizes the mutant signaling cascade enzyme with higher affinity than the wild-type signaling cascade enzyme. In particular embodiments, the mutant signaling cascade enzyme and the wild-type signaling cascade enzyme are protein kinases.

In some aspects, each of the peptide constructs of the first library of peptide constructs comprises a peptide portion and an identifying nucleic acid portion that identifies the peptide portion. The at least one peptide construct of the first library of peptide constructs bound to the target molecule is bound at its peptide portion to the target molecule. In some embodiments, the the identifying nucleic acid portion of each peptide construct comprises a polynucleotide sequence or complement thereof encoding the peptide portion of the peptide construct. In some aspects, the identifying nucleic acid portion of each peptide construct encodes at least 5 randomized amino acids, and the identifying nucleic acid portions are generated with full nucleotide randomization at the first and second positions of each of at least 5 randomized codons and G/T randomization at the third position to minimize stop codons and maximize synthetic yield.

In some aspects, the methods comprise separating the at least one peptide construct of the first library of peptide constructs bound to the first target molecule from the at least one peptide constructs of the first library of peptide constructs not bound to the first target molecule. Thus, in some aspects, the method further comprises immobilization and/or precipitation of the at least one peptide construct capable of specific binding to the target molecule using a capture agent having specific binding to the target molecule. For example, immunoprecipitation with an antibody or antigen-binding fragment having specific binding to the target molecule is used to separate the at least one peptide construct of the first library of peptide constructs bound the target molecule. The separating step may also comprise separating the peptide constructs based on differences in size after contacting the target molecule with the first library, for example, via filtration, centrifugation, size exclusion chromatography, or combinations thereof.

For embodiments of the methods where each of the peptide constructs of the first library of peptide constructs comprises a peptide portion and an identifying nucleic acid portion that identifies the peptide portion, the methods may further comprise sequencing all or a portion of the identifying nucleic acid portion of the at least one peptide construct of the first library of peptide constructs bound to the target molecule. For example, the sequencing step comprises amplification and next generation sequencing of the identifying nucleic acid portion. In some aspects of such embodiments, the method further comprises immobilizing the peptide portion of the variant construct with increased specific binding to the target molecule or with differential specific binding to the first and the second target molecules to a platform matrix or membrane to produce a diagnostic assay or detection kit. The peptide portion of the variant construct may be immobilized with an affinity tag/recognition entity interaction. For example, polyhistidine/NTA/Ni²⁺, glutathione S-transferase/glutathione, maltose binding protein/maltose, streptavidin/biotin, biotin/streptavidin, or antigen (or antigen fragment)/antibody (or antibody fragment). In certain implementations, the diagnostic assay or detection kit is a lateral flow assay.

### Z Score Calculations

The determination of Z scores as indicators of binding begins with grouping all of the peptides in the libraries into bins. Each bin is selected to represent a set of peptides present at very similar relative abundances in the starting peptide construct library. These bins are generated based on the measured relative abundances of peptides in a collection of negative control assays, which can consist of buffer-only controls with no target present or assays done with non-focal targets. Relative abundance of each peptide in each assay is calculated by dividing a peptide's read count by the total read count for the sample and then multiplying by 1 million. The result is reads mapped per million (rpm). For each peptide, the sum of the rpm values across all negative controls are calculated, and then the peptides are rank ordered from lowest abundance to highest. Peptides with similar rpm sums are placed in one bin, with each bin containing at least 300 peptides. If greater than 300 peptides have identical values, then all of those peptides are all assigned to the same bin. Otherwise, peptides with different, but similar values are combined in a bin until the minimum size of 300 peptides is reached. One of the benefits of using negative control assays to generate bins, instead of using the starting pool of peptides, is that in addition to accounting for differences in starting abundance, this approach also accounts for non-specific enrichment, as peptides that bind to reagents non-specifically will be present in these negative controls at higher relative frequencies than in the starting library.

Z scores are then calculated independently for each assay. In some implementations, the different assays all use the same set of negative control bins. The first step is again the calculation of relative abundances (rpm) for each peptide. These relative abundances are then further normalized (normDiff) by comparison to the average relative abundance in the negative controls (normDiffᵢ=rpmFocalᵢ−rpmNegControlAvgᵢ, where i represents an individual peptide in the assay). This normalization controls for small differences in relative abundance of peptides within a single bin. Z scores are then calculated separately for peptides in each bin using the normDiff values as input.

In order to calculate Z scores for a bin, the mean and standard deviation of normDiff values are calculated using the all of the peptides (at least 300) contained within the bin. It is important that these calculations do not include any peptides that have bound to and therefore been enriched by the target. This is because the mean and standard deviation are supposed to represent the distribution of expected values in the absence of binding to the target. Because it is generally expected that the true number of binders would be <5% of the total peptides within any bin, typically 5% of the values from each bin are removed prior to calculating these summary statistics. Preferably, the 5% of values that represent the most substantial outliers are removed. These substantial outliers are identified using the 95% highest density interval (hdi). Use of the hdi makes the calculations robust to differences in the shape of the underlying distribution. In cases where a larger percent of the peptides may be enriched, a larger percentage of the values can be removed prior to calculating the mean and standard deviation. Z score are then determined for each peptide by calculating the number of standard deviations away from the mean, with the standard deviation and mean being specific to the bin to which the peptide belongs. Therefore, the higher the Z score, the stronger the evidence of binding to the target and therefore enrichment of the peptide during the assay.

Accordingly, in certain implementations, the z-score of a peptide is calculated by first determining a relative abundance level of each peptide constructs in the library of peptide constructs and then grouping the grouping peptide constructs into bins based on similarity of relative abundance level, wherein each bin comprises at least 300 peptide constructs. The relative abundance level of each peptide construct is also normalized against the average of the relative abundance level of the negative control peptide constructs in the library of peptide constructs. The normalized relative abundance levels of each peptide construct in a bin are used to determine a mean and a standard deviation of each bin. The z-score of a peptide is calculated based on the mean and a standard deviation of its bin. In some aspects, the determination of the mean and the standard deviation of the normalized relative abundance levels in a bin excludes peptide constructs having outlier relative abundance levels. In some aspects, a peptide construct has an outlier relative abundance level when its normalized relative abundance level is outside the 95% highest density interval of its bin. In certain implementations, 5% of peptide constructs in each bin are excluded from the determination of the mean and the standard deviation of the normalized relative abundance levels in a bin.

### High Affinity Binary-Ligands Identified by Peptide-DNA Conjugate Libraries

In certain aspects, the present invention involves the use of the peptide-DNA conjugate methodology disclosed herein to improve ligand binding affinity by the cooperative binding of at least two lower affinity ligands.

It has been established by multiple research groups that significantly higher binding affinities can be achieved if two lower affinity ligands are molecularly linked to form a cooperative binding complex. If each ligand can bind to a unique target site, then the binding of the first increases the probability of the second binding. This is because the second binding event does not rely upon random diffusion in three dimensional space but, rather, has only a short and restricted path to bind. The two ligand complex is then stabilized by additional molecular interactions which are much stronger than those of the single ligands. In addition, if two linked ligands are bound, the disassociation of one ligand will require the dissociation of the second linked ligand to fully release from the target. Hence, there is a high probability that it will experience re-binding before the binary complex disassociates. Thus, the overall disassociation rate is lowered and contributes to an overall lower K_{d} for the binary ligand.

Peptide-DNA conjugate libraries can be designed to include small peptide ligands at either end of a larger peptide. This strategy would place one at the N-terminus and one at the C-terminus. A molecular peptide linker can be synthesized between the two ligands, linking them to allow cooperative binding kinetics. In order to seek the optimal spacing between the two ligands, the linker could be designed for various lengths. The exact amino acid sequence of the linker could vary, but glycine (G)-serine (S) polymers have been used successfully. For example, the repeated motif of (GGGS)ₙ, could be used to create short, intermediate, and even very long linkers to facilitate the search for optimal spacing. If n=1, the linker would be 4 amino acids long. If n=5, the linker could be 20 amino acids longer, etc.

Other linkers that can be used include, but are not limited to, linkers with the repeated motif of (GGGGS)ₙ, (e.g., (GGGGS)₃); linkers with repeated glycines only (e.g., (Gly)₆ and (Gly)₈); linkers with the repeated motif of (EAAAK)ₙ, (e.g., (EAAAK)₃); and poly(ethylene glycol) or PEG-linkers. Additional linkers that can be used are listed in Chen et al., Adv Drug Deliv Rev. 2013 Oct. 15; 65(10): 1357-1369.

In certain aspects of the invention, peptides identified singly as binders to a particular target are subsequently coupled in pairs to identify higher affinity combinations. The peptide-DNA conjugate method readily accomplishes this for even a moderately large number of ligands. For example, if 100 peptide ligands are known to bind to a target molecule then:
1) This represents only 10,000 combinations, even if each peptide is paired with itself and with every other ligand in both the C- and N-terminus locations; and
2) Each of these 10,000 combinations could be tested at four different linker lengths and it would still only represent 40,000 peptide species in the library.
   The inventors are routinely using and testing libraries exceeding this complexity. The best binding binary ligands would be determined though a combo-ligand library binding assay against the target, with deep next generation sequencing generating the quantitative profile of the high affinity binders. Because the individual ligands will be included in the library at both the C- and N-termini, these constructs can be used as internal standards to judge the binary ligands' affinity.

### Development of Peptidomimetic and Non-Peptidomimetic Therapeutic Agents

In certain aspects, the disclosed methods further comprise compiling a map of peptide constructs capable of specific binding to the target molecule to identify at least one binding site on the target molecule for targeting with a therapeutic agent. In some embodiments, target molecule is a protein kinase. In other embodiments, the therapeutic agent is a protein kinase inhibitor.

The human genome contains about 560 protein kinase genes, and they constitute about 2% of all human genes (Manning et al. (2002) Science 298 (5600): 1912-1934). Up to 30% of all human proteins may be modified by kinase activity, and kinases are known to regulate the majority of cellular pathways, especially those involved in signal transduction. The chemical activity of a kinase involves transferring a phosphate group from a nucleoside triphosphate (usually ATP) and covalently attaching it to specific amino acids with a free hydroxyl group. Most kinases act on both serine and threonine (serine/threonine kinases), others act on tyrosine (tyrosine kinases), and a number act on all three (dual-specificity kinases) (Dhanasekaran & Premkumar (September 1998). Oncogene. 17 (11 Reviews): 1447-55). Aberrant kinase signaling is associated with many diseases and conditions including cancer.

The discovery that specific molecular targets in cancer can be controlled with kinase inhibitor drugs revolutionized modern chemotherapy and created a new paradigm for drug development and treatment. Based on its blockbuster success, the kinase inhibitor drug imatinib (IM) (also known as Gleevec™), has become the first-line therapy for chronic myeloid leukemia (CML). IM targets the oncogenic kinase Bcr-Abl, the fusion protein resulting from the translocation of chromosomes 9 and 22 (known as the Philadelphia chromosome, the hallmark of CML), and initially induces remission in nearly all CML patients. A significant proportion of these patients (approximately 60-70%) maintain remission for ≥5 years (remarkable for a disease that previously had estimated 5-year survival rates of less than 50%).

In some embodiments, the target molecule is one or more kinases, including protein kinases of the following common families or subgroups: AGC (e.g., containing the PKA, PKG and PKC subfamilies), CAMK (e.g., calcium/calmodulin-dependent protein kinases), CK1 (e.g., casein kinase 1), CMGC (e.g., containing the CDK, MAPK, GSK3 and CLK subfamilies), NEK, RGC (e.g., receptor guanylate cyclases), STE, TKL (e.g., tyrosine protein kinase-like), and Tyr (e.g., tyrosine protein kinase). In some embodiments, the target molecule is one or more kinases of atypical kinase families, such as, ADCK, alpha-type, FAST, PDK/BCKDK, PI3/PI4-kinase, RIO-type, etc.

After identifying at least one binding site on the target molecule for targeting with a therapeutic agent, the therapeutic agent is designed based on the amino acid sequence from a peptide portion of a peptide construct with high affinity to the target molecule. In certain aspects, the amino acid sequence comprises at least 2 consecutive amino acids, at least 3 consecutive amino acids, at least 4 consecutive amino acids, at least 5 consecutive amino acids, at least 6 consecutive amino acids, at least 7 consecutive amino acids, at least 8 consecutive amino acids, at least 9 consecutive amino acids, or at least 10 consecutive amino acids from the peptide portion of a peptide construct with high affinity to the target molecule. In other aspects, the amino acid sequence comprises between 1 and 5 consecutive amino acids, between 1 and 10 consecutive amino acids, between 1 and 15 consecutive amino acids, between 1 and 20 consecutive amino acids, between 5 and 10 consecutive amino acids, between 5 and 15 consecutive amino acids, or between 5 and 20 consecutive amino acids from the peptide portion of a peptide construct with high affinity to the target molecule.

In some embodiments, the target molecule is an enzyme and activity of the enzyme is determined with and without the therapeutic agent to confirm the efficacy of the therapeutic agent. In one embodiment, the target molecule is a protein kinase, and the activity of the protein kinase is determined with and without the therapeutic agent.

### Lateral Flow Assays

As used herein, the term “analyte” is used as a synonym of the term “marker” and intended to minimally encompass any chemical or biological substance that is measured quantitatively or qualitatively and can include small molecules, proteins, antibodies, DNA, RNA, nucleic acids, virus components or intact viruses, bacteria components or intact bacteria, cellular components or intact cells and complexes and derivatives thereof.

The term “sample” as used herein refers to a volume of a liquid, solution or suspension, intended to be subjected to qualitative or quantitative determination of any of its properties, such as the presence or absence of a component, the concentration of a component, etc. Typical samples in the context of this application as described herein can include human or animal bodily fluids such as blood, plasma, serum, lymph, urine, saliva, semen, amniotic fluid, gastric fluid, phlegm, sputum, mucus, tears, stool, etc. Other types of samples are derived from human or animal tissue samples where the sample tissue has been processed into a liquid, solution or suspension to reveal particular tissue components for examination. The embodiments of the present application, as intended, are applicable to all bodily samples, but preferably to samples of whole blood, urine or sputum.

In other instances, the sample can be related to food testing, environmental testing, bio-threat or bio-hazard testing, etc. The foregoing, however, represents only a small example of samples that can be used for purposes of the present invention.

In the present invention, any determinations based on lateral flow of a sample and the interaction of components present in the sample with reagents present in the device or added to the device during the procedure and detection of such interaction, either quantitatively or qualitatively, may be for any purpose, such as diagnostic purposes. Such tests are often referred to as “lateral flow assays”.

Examples of diagnostic determinations include, but are not limited to, the determination of analytes, also referred to synonymously as “markers”, specific for different disorders, e.g., chronic metabolic disorders, such as blood glucose, blood ketones, urine glucose, (diabetes), blood cholesterol, (atherosclerosis, obesity, etc.); markers of other specific diseases, e.g., acute diseases, such as coronary infarct markers (e.g., tropinin-T, NT-ProBNP), markers of thyroid function (e.g., determination of thyroid stimulating hormone (TSH)), markers of viral infections (the use of lateral flow immunoassays for the detection of specific viral antibodies), cancer markers, etc.

Yet another important field is the field of companion diagnostics in which a therapeutic agent, such as a drug, is administered to an individual in need of such a drug. An appropriate assay is then conducted to determine the level of an appropriate marker to determine whether the drug is having its desired effect. Alternatively, the assay device usable with the present invention can be used prior to the administration of a therapeutic agent to determine if the agent will help the individual in need.

Yet another important field is that of drug tests, for easy and rapid detection of drugs and drug metabolites indicating drug abuse; such as the determination of specific drugs and drug metabolites in a urine or other sample.

The term “lateral flow device” as discussed throughout this application herein refers to any device that receives a fluid, such as sample, and includes a laterally disposed fluid transport or fluid flow path along which various stations or sites (zones) are provided for supporting various reagents, filters, and the like through which sample traverses under the influence of capillary or other applied forces and in which lateral flow assays are conducted for the detection of at least one analyte (marker) of interest.

The terms “automated clinical analyzer”, “clinical diagnostic apparatus”, or “clinical analyzer” as discussed herein, refer to any apparatus enabling the scheduling and processing of various analytical test elements, including lateral flow assay devices, as discussed herein and in which a plurality of test elements can be initially loaded for processing. This apparatus further includes a plurality of components/systems configured for loading, incubating and testing/evaluating a plurality of analytical test elements in automated or semi-automated fashion and in which test elements are automatically dispensed from at least one contained storage supply, such as a cartridge or other apparatus, without user intervention.

The term “testing apparatus” as used herein refers to any device or analytical system that enables the support, scheduling and processing of lateral flow assay devices. A testing apparatus can include an automated clinical analyzer or clinical diagnostic apparatus such as a bench, table-top or main frame clinical analyzer, as well as point of care (POC) and other suitable devices. For purposes of this definition, the testing apparatus may include a plurality of components/systems for loading and testing/evaluating of at least one lateral flow device, including detection instruments for detecting the presence of at least one detectable signal of the assay device.

The terms “zone”, “area” and “site” as used throughout this application define parts of the fluid flow path on a substrate, either in prior art devices or in at least one lateral flow assay device according to an embodiment of this invention.

The terms “reaction” is used to define any reaction, which takes place between components of a sample and at least one reagent or reagents on or in the substrate, or between two or more components present in the sample. The term “reaction” is in particular used to define the reaction, taking place between an analyte (marker) and a reagent as part of the qualitative or quantitative determination of the analyte.

The terms “substrate” or “support”, as used herein, refers to the carrier or matrix to which a sample is added, and on or in which the determination is performed, or where the reaction between analyte and reagent takes place.

The term “detection” and “detection signal” as used herein, refers to the ability to provide a perceivable indicator that can be monitored either visually and/or by machine vision, such as a detection instrument.

Components of the herein described lateral flow assays and lateral flow assay devices (i.e., a physical structure of the device whether or not a discrete piece from other parts of the device) described herein can be prepared from copolymers, blends, laminates, metallized foils, metallized films or metals. Alternatively, device components can be prepared from copolymers, blends, laminates, metallized foils, metallized films or metals deposited one of the following materials: polyolefins, polyesters, styrene containing polymers, polycarbonate, acrylic polymers, chlorine containing polymers, acetal homopolymers and copolymers, cellulosics and their esters, cellulose nitrate, fluorine containing polymers, polyamides, polyimides, polymethylmethacrylates, sulfur containing polymers, polyurethanes, silicone containing polymers, glass, and ceramic materials. Alternatively, components of the device can be made with a plastic, elastomer, latex, silicon chip, or metal; the elastomer can comprise polyethylene, polypropylene, polystyrene, polyacrylates, silicon elastomers, or latex. Alternatively, components of the device can be prepared from latex, polystyrene latex or hydrophobic polymers; the hydrophobic polymer can comprise polypropylene, polyethylene, or polyester. Alternatively, components of the device can comprise TEFLON®, polystyrene, polyacrylate, or polycarbonate. Alternatively, device components are made from plastics which are capable of being embossed, milled or injection molded or from surfaces of copper, silver and gold films upon which may be adsorbed various long chain alkanethiols. The structures of plastic which are capable of being milled or injection molded can comprise a polystyrene, a polycarbonate, or a polyacrylate. In a particularly preferred embodiment, the lateral flow assay devices are injection molded from a cyclo olefin polymer, such as those sold under the name Zeonor®. Preferred injection molding techniques are described in U.S. Pat. Nos. 6,372,542, 6,733,682, 6,811,736, 6,884,370, and 6,733,682, all of which are incorporated herein by reference in their entireties.

The present invention is further illustrated by the following examples that should not be construed as limiting. The contents of all references, patents, and published patent applications cited throughout this application, as well as the Figures, are incorporated herein by reference in their entirety for all purposes.

### EXAMPLES

### Example 1

### Rapid Design and Production of Small Molecule Libraries Using the Peptide-DNA Conjugate Technology

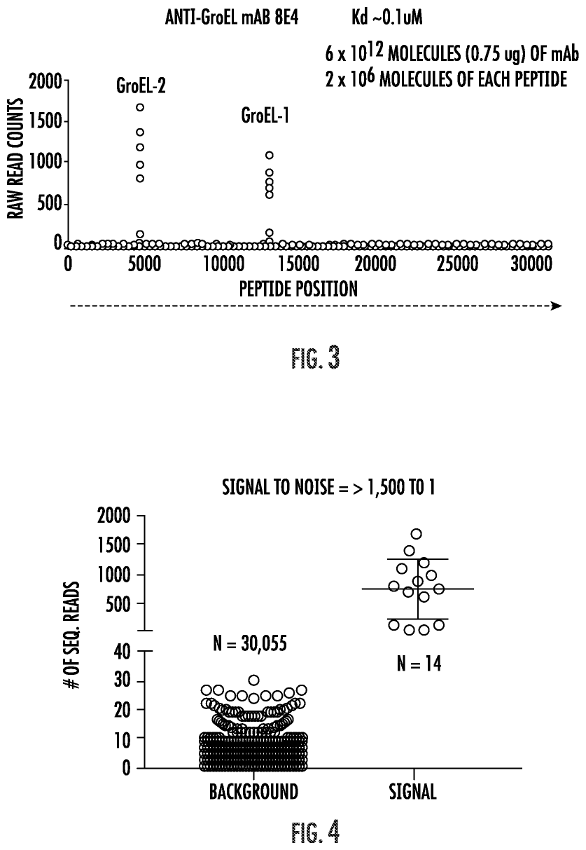

The peptide-DNA conjugate approach is a fully in vitro method for generating large libraries of peptides, each of which is conjugated to a unique DNA tag that identifies it by next generation sequencing. The inventors have been using this technology extensively to identify immunological epitopes to monitor serological responses to bacterial, viral, and fungal diseases. The inventors have built 15 genome-based libraries of 30,000-244,000 peptides and used them in both antibody and MHC II binding assays. The peptide content is specified through custom synthetic oligonucleotides, which are the starting point for the generation of libraries. These oligonucleotides are then in vitro transcribed/translated to generate the final product of a peptide-DNA conjugates (see FIG. 2).

### Example 2

### Identification of Specific Peptides from the Libraries that Bind to a Biological Target Using the DNA Tags

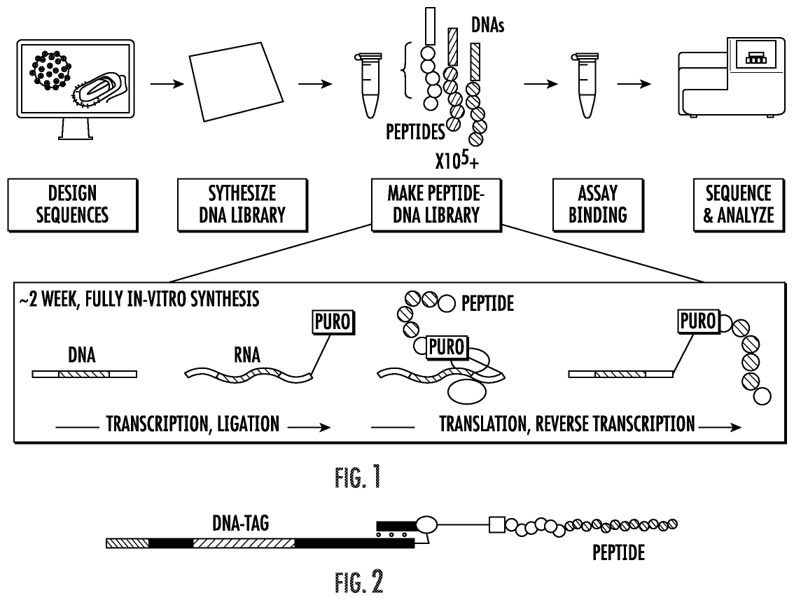

The inventors designed previous peptide libraries from pathogen genomes in order to explore the pathogen encoded epitopes that stimulate host responses. These libraries contain 30,000-244,000 peptide-DNA conjugates, which the inventors then assayed for binding against antibodies or MHC II molecules. The overall process outlined in FIG. 1 includes binding, pull down, and bulk next generation sequencing of the DNA-tags connected to the bound peptides. See Kozlov et al., PLoS One. 2012; 7(6):e37441. The basic structure of the DNA-peptide conjugates used in this process is presented in FIG. 2.

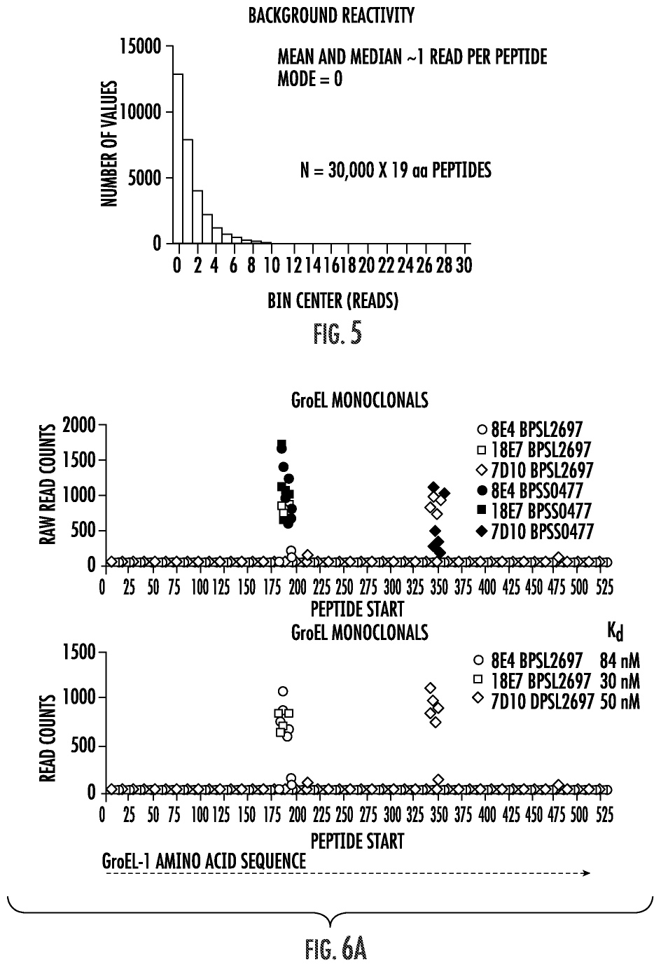

FIGS. 3-6 present the results of one such set of experiments where peptides designed from the Burkholderia pseudomallei genome were bound (or not) by a panel of monoclonal antibodies (mAbs). This proof of principle demonstrates that binding assays can be developed in order to identify rare ligands within complex pools that interact with a biological target molecule. The peptide-DNA conjugate method qualitatively identifies which peptides were bound, but also quantifies this in a manner determined by the number of binding sites and their affinities.

The specific mAb 8E4 binding assay parameters were as follows:

Bₘₐₓ=6×10¹² molecules
0.75 μg of mAb
2 binding sites on each mAb
10 μL volume
Individual Peptides=2×10⁶ molecules

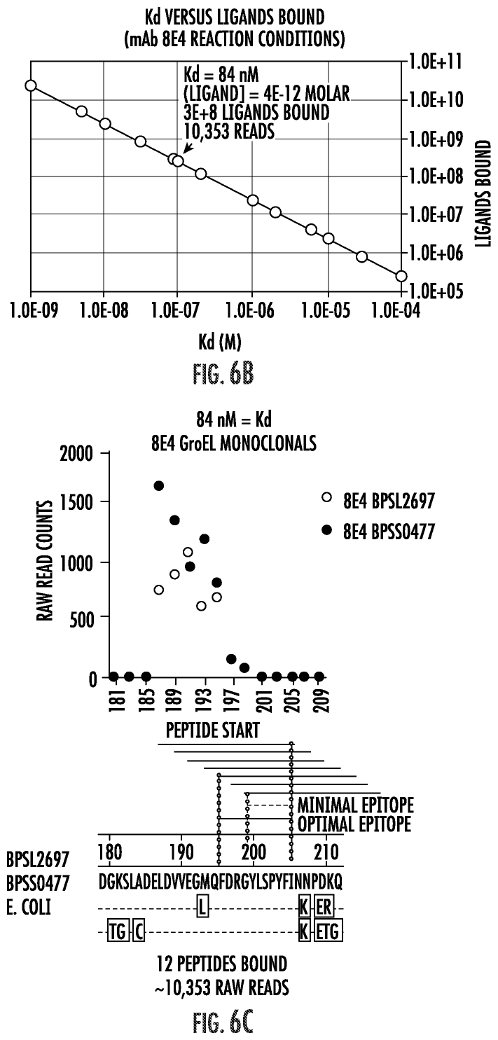

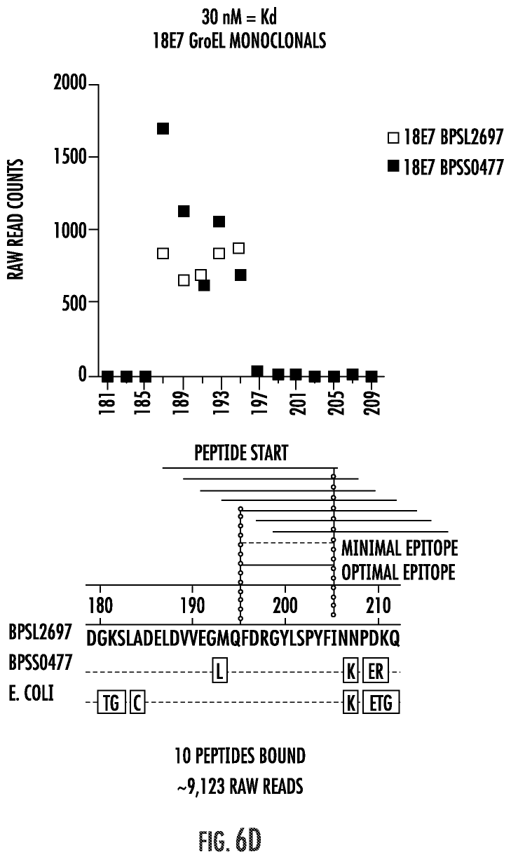

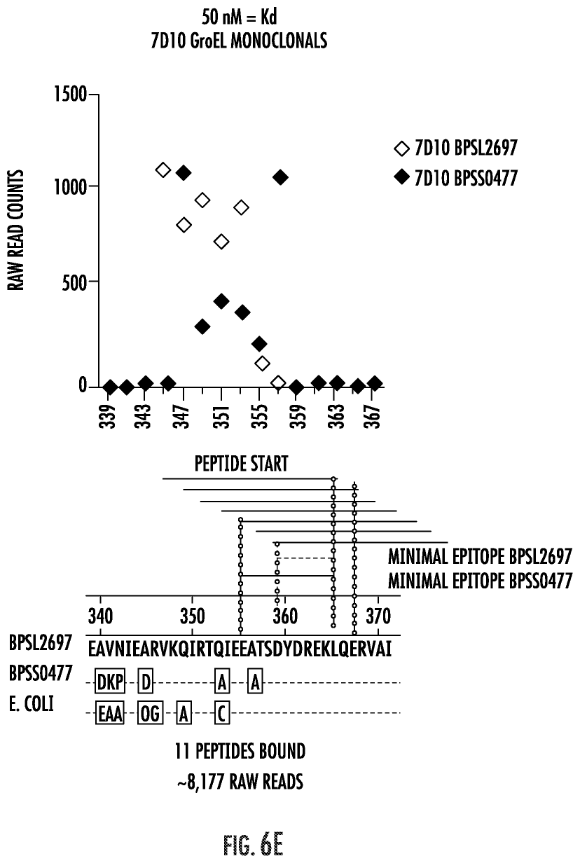

Reaction volume=10 μL
Concentration of individual peptide=3.3E-13 Molar
Concentration of 12 combined peptides=4.0E-12 Molar
Burkholderia peptide-DNA conjugate library complexity 30,000
190 Burkholderia genes with sliding window (2 aa) design
Uncorrected for synthesis bias From the complex Burkholderia peptide-DNA conjugate library, the anti-GroEL mAb 8E4 recognized those peptides encoded by particular sections of the GroEL-1 and GroEL-2 genes (see FIG. 3). Surprisingly, in such a complex mixture of peptides the signal to noise ratio was greater than 1,500 to 1 (see FIG. 4). In addition, the background reactivity of other peptides in the Burkholderia peptide-DNA conjugate library was quite low with the assay (see FIG. 5). The assay was expanded to include the anti-GroEL mAbs, 18E7 and 7D10, along with 8E4. The K_{d} of each of 8E4, 18E7, and 7D10 was determined to be 84 nM, 30 nM, and 50 nM, respectively (see FIG. 6A). Only 525 peptides from the GroEL gene itself are shown in FIG. 6A (X-axis), but the full library contained 30,000 unique peptides. The Y-axis is the sequencing read count for each peptide. As shown in FIG. 6B, as the K_{d} value decreases the number of ligands bound and the related signal in the assay (i.e., raw read counts) increase.

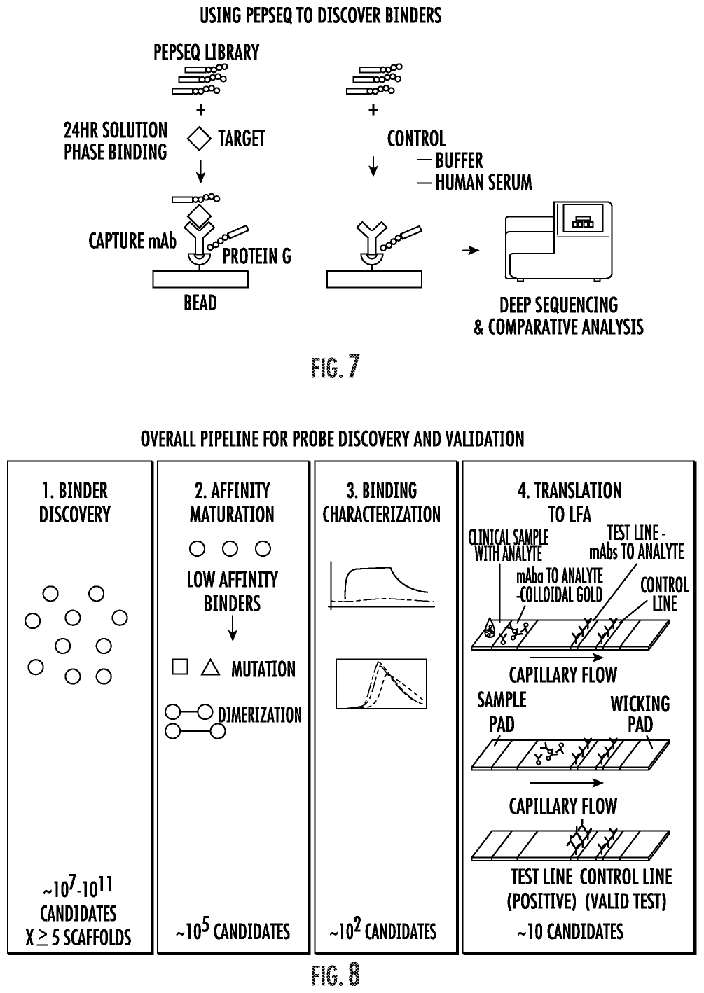

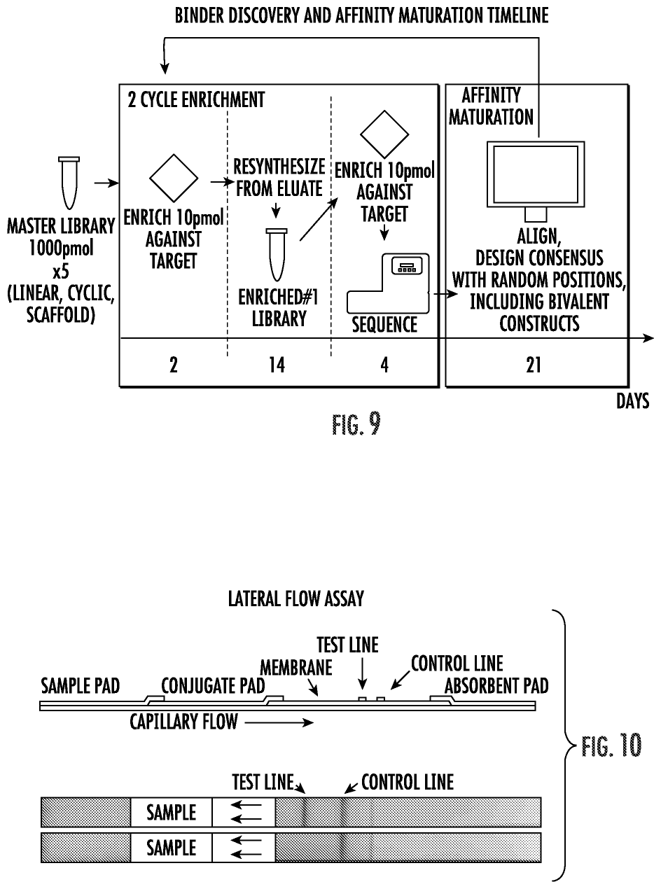

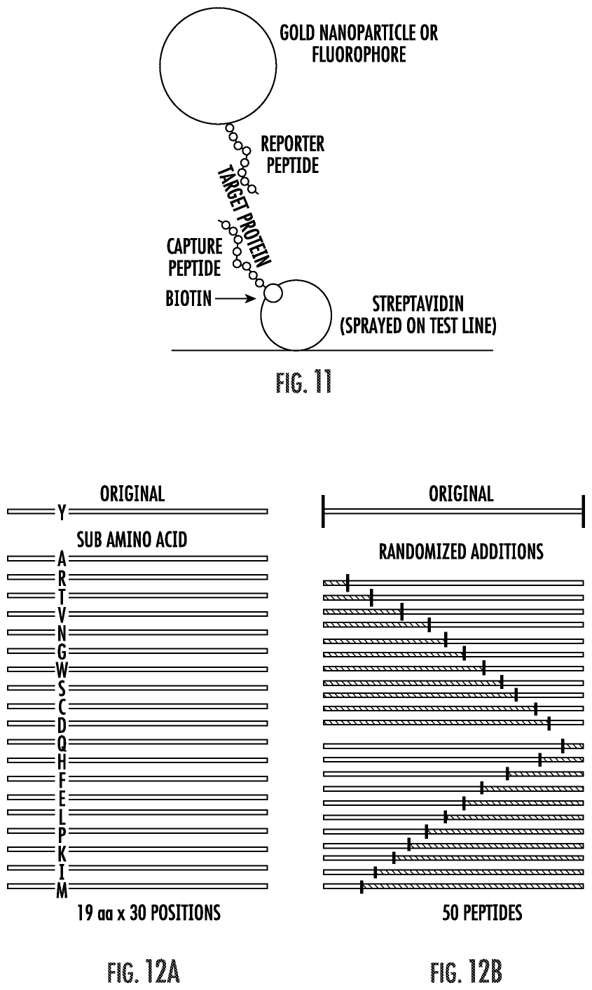

Analysis of the peptides producing the highest signals allowed for the identification of the minimal epitope and the optimal epitope sequences for each of the anti-GroEL mAbs, 8E4, 18E7 and 7D10 (see FIGS. 6C-6E). A similar approach is used when using a peptide-DNA conjugate library to identify binders for a target as shown in FIGS. 7-8. During binder discovery, the initial binder candidates are identified and amino acid sequences shared between candidates are analyzed to determine consensus sequences. During affinity maturation, the initial candidates are re-screened together with variant peptides with mutations in the consensus sequences previously identified. Dimerization of low affinity binders is also evaluated to identify binders with higher affinity. An example of a timeline for binder discovery and affinity maturation is presented in FIG. 9.

With each subsequent screen, binding of candidate peptides from the modified library is characterized. Finally, the binders with the desired sensitivity and specificity are used to develop a detection assay (e.g., an LFA) or a therapeutic agent (see FIG. 8 for an overview of the process).

### Example 3

### Exploring the Chemical Space Surrounding a Candidate Molecule to Optimize the Molecular Structure for Optimal Binding

Once a candidate peptide is identified, a subsequent peptide-DNA conjugate library can be used to generate many variants to define the binding moiety and identify higher affinity variants. In the GroEL example, the inventors used a sliding window along the protein sequence to find the best binding moiety, which is smaller than the full length peptide. This strategy can be expanded to include amino acid substitutions adjacent to the candidate peptides to identify higher affinity variants.

### Example 4

### Attachment of Small High Affinity Molecules to a Platform Matrix for Assay Development

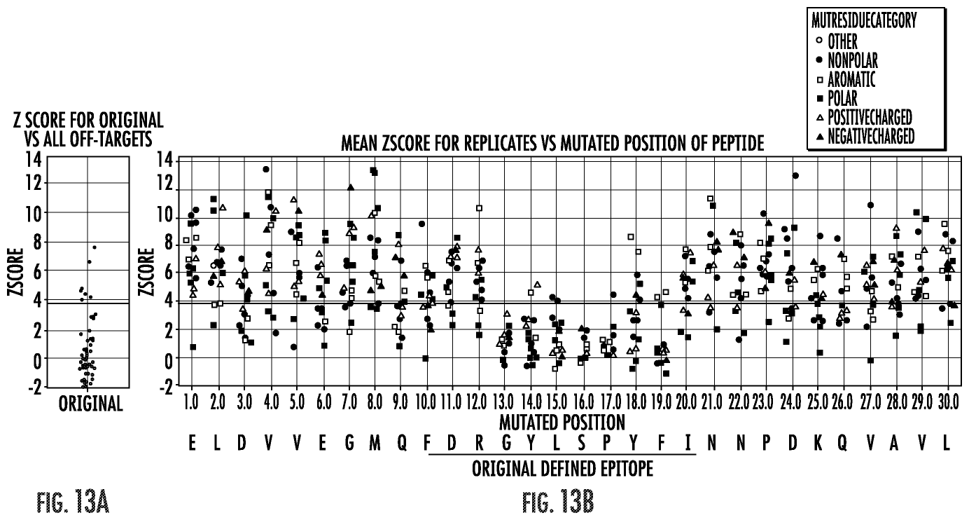

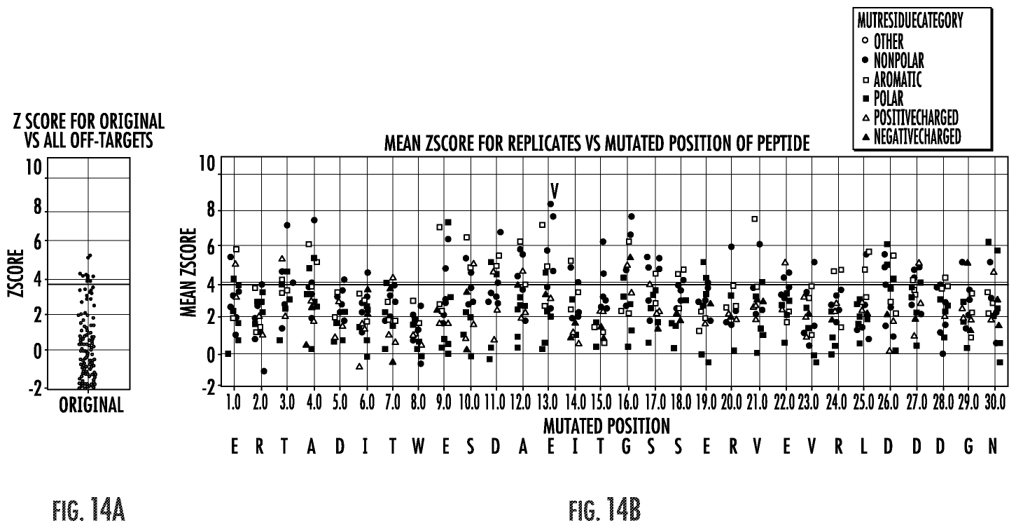

Because the SYMBA molecules are all peptides, the chemistry for their attachment to matrices is well established for both the C- and N-termini. In the serological studies, the inventors have manufactured particular high-affinity peptides commercially and then attached them to LUMINEX® Assay beads. These beads have been successfully used in assays to detect antibodies on the MAGPIX® platform. Alternatively, the capture peptides can be attached to nitrocellulose strips and the reporter peptides to gold particles or fluorophores. In one example, the capture peptide is engineered with a tag (e.g., biotin) that is bound to a platform matrix or membrane (e.g., a nitrocellulose strip) in a lateral flow assay (e.g., with streptavidin) as shown in FIG. 10 and FIG. 11.

### Example 5

### Design and Production of Highly Diverse DNA-Barcoded Small Molecule Libraries Using Peptide-DNA Conjugate Technology

The inventors designed a library which contains 7 amino acid positions that are fully-randomized within its structure. This library represents a diversity of ˜10⁹ unique molecules, and present the 7 randomized amino acids with different 3D configurations. While the inventors can easily increase the diversity, the 10⁹ diversity was selected to complement the 10 pmol (˜6×10¹² molecules) of library used per binding assay. Hence, each peptide species will be present 6000 times.

The first and simplest library comprise a simple randomized 7 mer flanked by spacer glycines and two cysteine residues that allow an inducible basal disulfide bridge. This library is assayed under both oxidizing and reducing conditions to generate both circular and linear structures. Additional libraries were designed using previously described constrained-conformation short polypeptide scaffolds (see Hosse et al., Protein Sci. 2006 January; 15(1):14-27) comprising 15-40 total amino acids each, in which the inventors engineer the 7 randomized amino acids at defined contact positions.

All of the libraries were synthesized starting from their own oligo template library, using full nucleotide randomization at the first and second positions of each of the 7 randomized codons, and G/T randomization at the third position (aka, an NNK strategy; see Barbas et al., Proc. Natl. Acad. Sci. USA 90:10003-10007, 1993), to minimize stop codons and maximize synthetic yield. The inventors are currently able to design & synthesize oligonucleotides, in vitro transcribe/translate, and QA/QC the libraries in a relatively short timeframe.

### Example 6

### Development and Optimization of Peptide-DNA Conjugate Library Binding Assays

The inventors have performed peptide-DNA conjugate library binding assays against antibodies (polyclonal and mAb) as well as against a panel of MHC II molecules. This involves mixing the libraries with the targets and then physically separating the unbound peptides from those that are bound through immunoprecipitation or other physical separations. The unique DNA tags from the bound fraction are PCR amplified, in bulk, and subjected to next generation sequencing to identify and quantify the binders (see FIGS. 3-6). The inventors have developed similar binding assays for bacterial and viral proteins. In these assays, bound vs. unbound peptides will be separated by immunological pull downs, streptavidin x biotin, or size separations.

### Example 7

### Screen of Random Small Molecule Libraries for Candidate Binders

Using the libraries from Example 5 and the binding assays from Example 6, the inventors screened the libraries for high affinity binding peptides against the selected targets. The inventors have a well-developed next generation sequencing pipeline for analyzing the bound components that includes software to differentiate binding signal to noise in a large data set (see Examples 11 and 12). Following this computational analysis, the candidate peptides were characterized for similarity (sequence and physical parameters) to potentially identify those potentially binding the same site. Likewise, similar peptides with large binding differences are used to predict the structural aspects dictating binding affinity. The diversity and quantitative metrics were used to prioritize candidates, with 100-1000 being used for subsequent development and screening.

### Example 8

### Design of Targeted Small Molecule Libraries to Optimize Binding of Candidates

The inventors explored the chemical space surrounding the identified SYMBA candidates to fine-tune binding. This was accomplished through the design and production of a lower complexity library where each peptide species is present >10⁶ in 10 pmol of library reagent. The inventors randomized amino acids flanking the identified binding motifs and explored structures in which several identified binding motifs were linked together in one polypeptide separated by various spacer sequences, in order to identify higher affinity variants. Because of the high capacity of the peptide-DNA conjugate libraries, this can be accomplished simultaneously for the thousands of candidate molecules. These are lower complexity libraries that serve to validate the initial binding observations prior to more focused development efforts.

The inventors also explored the use of binary-ligand capture and reporters with the peptide-DNA conjugate library method to increase the affinity of the reagent. Once single candidate peptide binders are identified, they were paired in single longer peptides with one peptide ligand on the C-terminus and one on the N-terminus. They were then separated by oligo (GGGS)ₙ, linkers, for example, of different lengths, with each labeled with a unique DNA tag. The peptide-DNA conjugate binding assay was used to select highest binding combo-peptides with the results based upon next generation sequencing of the associated DNA tags. Two linked ligands will likely significantly increase the affinity, sensitivity, and specificity of the resulting LFA. It is applicable to both the reporter and capture SYMBA.

### Example 9

### Characterization of the Binding Affinity and Specificity of Selected Small Molecules

As the candidate peptides were identified and improved, their suitability for inclusion in the prototype LFA was judged by their binding affinity to the target. This was determined using a BIACORE™ surface plasmon resonance (SPR) instrument. The inventors determined K_{d} measurements for the peptides specific to the targets. The best candidates were tested in combinations on the BIACORE™ to detect interference effects for potential reporter and capture peptide combinations. Binding measurements on the BIACORE™ flow cell better represent the fluid flow than equilibrium-based methods (e.g., ELISA). The binding affinity studies were carried out to judge the potential of candidate peptides for further development. This is an essential and last step before moving the candidate peptides into the LFA development phase.

### Example 10

### Development of a Prototype LFA Based Upon the High Affinity Small Molecules

The inventors developed an LFA format that is generic and functions for peptide SYMBA-based assays against various targets. This involved the use of a universal reagent capture line that binds to an engineered constant feature of the capture peptide (e.g., biotin, his-6 tag, or other affinity linker). The capture peptide is pre-incubated with the target as well as the gold-labeled reporter peptide, and then loaded onto the LFA. The tertiary complex then binds to the generic capture strip on the nitrocellulose (see FIGS. 10-11). Hence, the nitrocellulose strips from the prototype LFA will be compatible with future assays involving peptide-based capture molecules. This represents a development feature that reduces the cost and accelerates the availability of future SYMBA-based assays.

The inventors optimized and validated the universal peptide capture line strategy. This validation was based upon small peptides that the inventors have previously shown to bind to mAbs against GroEL (see FIGS. 3-6). The peptides were modified with his-6, biotin, or other affinity tags and then tested against a series of anti-GroEL mAbs on the LFA format. Nitrocellulose strips were line sprayed with anti-his6 mAbs or streptavidin to create a universal capture line. The peptide and mAb were combined in a tube then added to the LFA sample pad. The complex flowed into the conjugate pad of the LFA where a gold-labeled anti-mouse reporter mAb bound the GroEL mAb bound to the peptide. The complex then flowed into the nitrocellulose and within minutes was captured by streptavidin on the test line binding to the avidin tag on the peptide.

In a similar fashion, a flipped assay assesses the performance of gold-conjugated reporter peptides. mAbs known to bind these same peptides were test line sprayed onto the nitrocellulose. The gold-reporter-conjugated peptides were added to the sample pad and allowed to flow into the nitrocellulose until captured by the GroEL-specific mAb. This strategy separately allows for optimization and validation of the proposed capture and reporter peptide functions.

The inventors conjugated candidate reporter peptides to gold particles and candidate capture peptides to nitrocellulose. The test line application of capture peptides to nitrocellulose and conjugation of gold particles to reporter peptides was optimized using several different strategies. The capture peptide were attached through simple line spraying, which works well for larger proteins (e.g., antibodies). This is simple but the small size of the peptide may affect binding performance at the test line. A second strategy was used to conjugate the peptide via amine chemistry to a carrier molecule such as bovine serum albumin (BSA), and then line spray it onto the membrane.

Finally, the inventors modified the capture peptide with a terminal 6x-his tag or biotin to allow for capture on the test line by streptavidin or anti-6x-his mAb, for example. The reporter peptide was directly conjugated to the gold particle. An alternative strategy involves the addition of a linker peptide that is conjugated to the gold particle by amine chemistry.

The inventors tested the LFA for limit of detection (LOD), sensitivity and specificity. The LFA was then fabricated at a larger scale and subjected to further testing. Fabrication equipment includes Biodot XYZ 3050 dispensing system, a Biodot guillotine strip cutter and a Biodot laminator. Densitometers were used for scanning of results from LFA assays.

This experimental work demonstrates the ability of the disclosed methods to identify high-affinity small molecules and then utilize them on a well-established detector platform, the Lateral Flow Assay. The inventors targeted bacterial and viral proteins to show the flexibility, speed, and cost effectiveness of this approach to field a simple and deployable LFA.

As outlined, the inventors sequentially screened high-diversity DNA-barcoded polypeptide libraries to identify the rare moieties that will bind to the target proteins. The target proteins can be produced in mammalian cells to allow for native glycosylation modifications.

The small peptide molecule libraries represent >1 billion unique molecules, from which the binding assay uses next-generation sequencing to identify the subset that bind strongly to the targets. The libraries are based upon short custom-designed polypeptides (e.g., 7 amino acids) that are produced by an in vitro transcription/translation process. Each library can be produced using a highly-controlled and fully in-vitro process at a very low cost. The speed and cost is critical as it allows an iterative design strategy, in which the inventors first screen highly diverse libraries for candidate molecules, and then perform a round of focused binder optimization. The initial libraries are target agnostic, while the secondary libraries are agent-specific and used to precisely define the binding moiety and related chemical space to identify higher affinity molecules. The inventors also use peptide-DNA conjugate libraries to test peptide combinations in order to identify binary ligands with higher affinity to the various targets. Importantly, once high affinity binding peptides are identified, they can be readily produced in a commercial GMP manufacturing facility prior to inclusion in the Lateral Flow Assay (LFA). The inventors developed multiple high affinity molecules to allow sandwich-type assays.

The inventors have developed a simple, yet innovative universal LFA format to generate a universal capture strategy for the prototype that is readily applicable for any future LFAs. The assay capture peptides are engineered with an affinity tag to the universal capture line. This allows for the production of a universal LFA where only the analytes are modified to match the targeted agent. The LFA technology is common and the basis for many FDA approved diagnostics.

### Example 11

### Identification of Peptides that Bind to a Target Protein Using Complex Peptide-DNA Conjugate Libraries

Complex PepSeq libraries can be used to identify particular peptides that bind to a target protein. This is accomplished by incubating the library with the target, which is bound to magnetic beads to allow its physical separation from unbound portions of the library. The bound versus unbound peptides are identified by difference in their abundance in the bound versus starting library. Due to uneven library manufacturing efficiencies, the starting abundances vary, and this makes identifying enriched bound peptides more difficult. The inventors use multiple negative controls as comparators for the specifically bound peptides versus non-specific background. In addition, in complex highly diverse libraries, individual peptides are at low abundances, making enrichment differentials hard to quantify.

Therefore, the inventors use a bin-based Z-score methodology that compares relative abundance estimates of groups of peptides known to be present at similar starting frequencies in the peptide-DNA conjugate library. Peptides are assigned to bins based on their relative frequency estimates in multiple negative control assays. Each bin contains ≥300 peptides with similar average relative frequency estimates across the negative controls. Peptides within a bin, therefore, are inferred to be present at similar relative abundances in the starting peptide-DNA conjugate library.

In order to account for slight differences in starting abundance between peptides contained in the same bin, relative abundance in experimental samples is first normalized to the corresponding value for the negative controls. These normalized abundances are then used to calculate a Z-score for each peptide within each sample. Each Z-score corresponds to the number of standard deviations away from the mean, with the mean and standard deviation calculated independently for the peptides from each bin. It is important that the mean and standard deviation reflect the distribution of unenriched peptides within a bin. Therefore, these calculations are based on a subset of the peptides contained in each bin. For assays with a low percentage of enriched peptides expected, the inventors typically use the 95% highest density interval of normalized counts within each bin.

### Example 12

### Lower Complexity Discovery Library Binders Confirmation and Maturation Using Fully Defined Libraries

Previously, two different lower complexity libraries were used to identify peptides that bind to the Bh and CHIKV targets. These lower-complexity libraries are composed of 244K 30 amino acid peptides, but the content of these peptides can be directed to produce the peptides of interest. The two libraries employed used three different strategies. The first was an existing Pan Viral (PV1) library designed and synthesized for other projects. The PV1 library is a 244K 30 mer peptide library that attempts to cover 148,215 proteins from 443 species-level viruses known to infect humans. The second library produced was called SYM1 and used two different discovery strategies. The first set is a random 5 mer peptide lower-complexity discovery library that is titled across a 30 amino acid peptide. By combining the random 5 mers across the 30 amino acid position, the per peptide concentration of the 5 mer was increased by >5000 fold compared to the high-diversity discovery libraries and >13 fold when using a traditional unique peptide sequence with no overlap. This overlapping strategy generated 3.2 million unique random 5 mer peptides that covers all of the potential 5 mer diversity within 144K 30 mer peptides. Another set of peptides was designed to explore the “interactome” of protein targets. The idea for this exploratory approach came from the COVID19 pandemic where a new virus was introduced but many of the interactions of the proteins of this virus were either quickly discovered or already known from related proteins (i.e., Spike and ACE2). To this end, the inventors went into the literature and identified proteins that were known to interact with the targets of interest. 30 mer peptides were generated using a 1-step tiling across the interacting proteins. These peptides used 50K peptides to investigate this potential interaction space.

Using these different libraries and a previously described enrichment strategy, the inventors identified a set of peptides that interacted with Bh virB5, Bh trwG, and CHIKV E1/E2. These peptides were then investigated further using another directed designed library, called SYM2, to confirm binding and also potentially “mature” the peptide binding affinity. An overview of this design is shown in FIG. 12. There are three different levels incorporated into this library based on the Z score-based priority of the original peptide. These modifications include: 1) Complete single residue mutagenesis —each residue is changed to all of the other possible 19 amino acids (aa) (total peptides used 19 aa×30 positions=570 peptides per original binder). This method would decrease binding through alanine and other residues but might also increase binding through other substitutions. 2) Sliding window—removal of N or C terminal portion of the peptide and replacement of random sequence (total peptides used 50 peptides per original binder. The goal is to define specific internal sequences that contribute to binding. This method can be compared to the mutagenesis to identify key regions. 3) Alanine scan—mutagenesis of the original binder to include alanine or glycine (total peptides used 30 per original binder). The goal is to identify important amino acids in binding but in a higher throughput method. Method 1 and 2, the complete single residue mutagenesis and sliding window methods, were applied to higher priority binders that the inventors wanted to confirm and mature and method 3, the alanine scan, was applied to binders that the inventors wanted to confirm only.

The SYM2 library peptides were selected based on Z score cut-off base on comparing the target to the other off-target enrichments. For full maturation, there were 245 peptides specific to virB5, 134 peptides specific to E1/E2, and 2 control peptides specific to the GroEL mAb. For the confirmation only (alanine scan), there were 72 peptides specific to VirB5, 153 peptides specific to trwG, and 9 peptides specific to E1/E2. Thus, a total of 613 potential binders were investigated to confirm binding and, in some cases, they were matured further. Using these peptides, the inventors designed peptides, determined nucleic acid codon sequences, ordered DNA, and synthesized peptide-DNA conjugate libraries. The synthesis of SYM2 yielded 281 pmol of library, which was sufficient to perform the necessary confirmation enrichment at 1 pmol of library per binding.

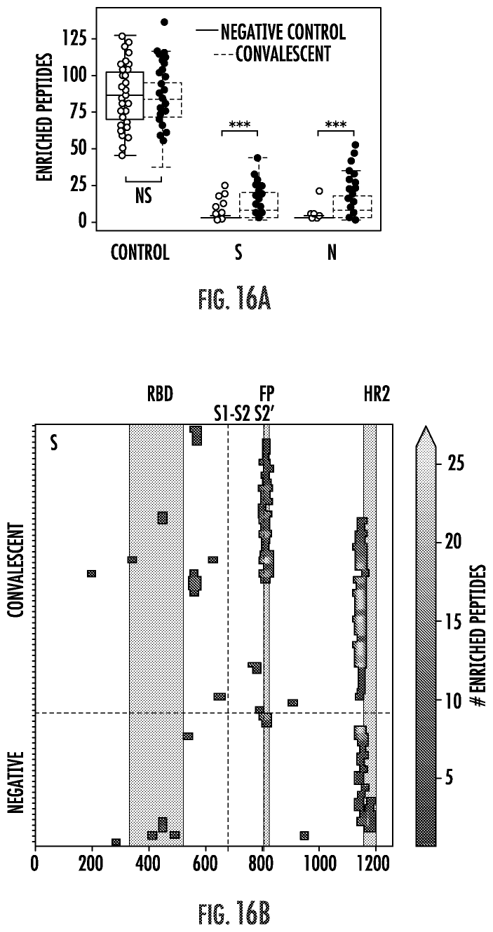

Initially, a control binding reaction using the GroEL monoclonal antibody (mAb) binding to a previous GroEL peptide (Burk_A_13002) containing a linear epitope was performed. These peptides and the interaction with the mAb were included to illustrate signal decrease when important regions are changed through confirmation mutagenesis since the required linear epitope for the 8E4 mAb was previously mapped and would be required for binding. In addition, changes to the linear epitope region or flanking regions that could result increased binding affinity could also be mapped. For these enrichments, the library was bound to 8E4 mAb, 7D10 mAb (comparison control), and a no protein control (comparison control). 6 replicates per enrichment were performed for a total of 18 binding reactions. Half of the samples were washed with cold buffer or room temperature buffer. This approach used 18 pmol of the SYM2 library. All bound peptides were eluted, and DNA tags were indexed and sequenced using the Illumina NextSeq instrument. Read counts were normalized between samples and Z-score comparisons were generated using the replicates for each enrichment. A Z-score of a peptide interaction with a target protein represents the number of standard deviations away from a comparison interaction mean. The resulting Z-score analysis for specific and non-specific binding is shown in FIG. 13A. In this binding assay, the peptide binds specifically to the 8E4 mAb (blue dots) and is separated from the non-specific interactions (grey dots). When amino acids are substituted to the other 19 possible amino acids at each position (FIG. 12B), regions required for binding are identified by a general decrease in signal (positions 13 through 19), highlighting the importance of this sequence for mAb binding. Specifically, no matter what amino acid is substituted at positions 13 and 16, there is a decrease in peptide binding to the mAb. However, certain substitutions do not decrease binding such as substitutions as position 15 and these substitutions that did not decrease signal are amino acids that have similar properties (i.e., nonpolar to nonpolar). In addition, some amino acid substitutions at position 18 resulted in an increase in Z-score signal and might indicated an increase in peptide:mAb binding affinity. These changes may stabilize the linear epitope and make it more likely for the mAb binding or increase the affinity of the epitope to the monoclonal antibody. Finally, substitutions to other regions outside of the linear epitope increased the Z-score of some peptides compared to the original peptide. Specifically, changes at position 4 and 8 resulted in the greatest change in signal compared to the original peptide. These changes may increase the availability of the linear epitope for antibody binding or may bind to other sites on the mAb. Further studies to correlate the change in Z-score and affinity are underway.

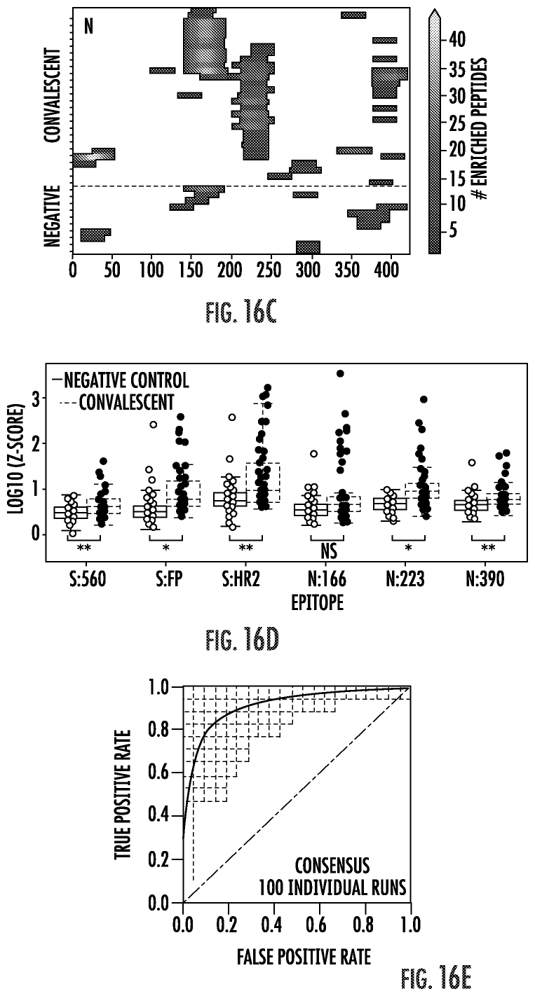

The inventors then bound the SYM2 library to trwG, VirB5, E1/E2, two additional control targets, and a no protein control. For each target the inventors performed a total of 4 replicates and samples were washed with room temperature buffer. This approach used a total of 24 pmol of SYM2 library. All bound peptides were eluted, and DNA tags were indexed and sequenced using the Illumina NextSeq instrument. Read counts were normalized between samples and bins for Z-score comparisons were generated using the replicates for each enrichment. The results of peptide PV1_079508 family of peptides is shown in FIGS. 14A-14B. The original peptide bound specifically to the virB5 target with an average Z-score of 4 (blue line). This signal can be thought of sensitivity of binding. In contrast, the non-specific interactions were lower than the specific interactions and had Z-scores ≤2 and this interaction and can be thought of the peptide specificity. Substitution of the amino acids at positions across the peptide resulted in both increases and decreases in Z-scores relative to the average virB5 Z-score of the original peptide (FIG. 14B). Substitutions at positions 2, 5-8, 18, and 28 had a similar or lower Z-score compared to the original peptide. This data suggests that these positions may be required for binding and any change in this sequence results in no change or a decrease in binding affinity. However, substitutions at several positions resulted in an elevated Z-score compared to the original peptide. An example is shown at position 13 where the glutamic acid (E) was substituted to a valine (V) or other nonpolar amino acids. This data suggests that certain positions can be changed, and these changes may result in a higher affinity interaction of the peptide with the virB5 target.

### Example 13

### Identification and Characterization of Specific Binding or Differential Binding Epitopes

### Peptide-DNA Conjugate Library Design

The inventors designed two different libraries of peptides in order to assess antibody reactivity to SARS-CoV-2 peptides and to peptides from other human-infecting CoVs. For the human virome (‘HV’) peptide library, the inventors sought to include sequences from all viruses known to infect humans. For viruses with RNA genomes, the inventors obtained a list of 214 virus species (see Woolhouse and Brierly, Sci Data, 2018; 5, 180017). NCBI taxonomy IDs were obtained for each of these species using the “names.dmp” file from the NCBI “new_taxdump” downloaded on Nov. 19, 2018 [note: “Bovine viral diarrhea virus 1” (NCBI:txid11099) was replaced with the corresponding species, “Pestivirus A” (NCBI:txid2170080)]. Taxonomy IDs for human viruses with DNA genomes were obtained using the “host.dmp”, “nodes.dmp” and “fullnamelineage.dmp” files from the NCBI “new_taxdump” downloaded on Nov. 26, 2018. In total, the inventors identified 289 taxonomy IDs annotated as virus species with DNA genomes that are known to cause human infections; however, 31 of these were excluded from the library design because they clearly belonged to unclassified adenovirus strains, rather than distinct virus species. Finally, the inventors included two taxonomy IDs associated with the Jingmenvirus group, members of which have recently been associated with human infections in China.

The inventors downloaded all viral protein sequences from the UniProt Knowledgebase on Nov. 19, 2018 and extracted the sequences annotated with one of the 474 target species taxonomy IDs. NCBI BLAST was used to identify sequences with non-viral components (i.e. recombinant), specifically those containing common reporter and therapeutic proteins: ubiquitin, luciferase, green fluorescent protein, chloramphenicol acetyltransferase, LacZ, GusA and GusB. These sequences were excluded from the assay design. To identify taxonomically misclassified proteins, the inventors downloaded all of the proteins annotated in the NCBI RefSeq database for the target species, when available (342/474 target species IDs). NCBI BLAST was then used to identify the best matching RefSeq protein for each UniProt protein, and flagged instances when the top hit was “strong” and to a RefSeq protein from a different genus (≥80% nt identity) or species (≥95% nt identity). All of the flagged UniProt proteins were manually investigated, including an additional BLAST to the NCBI nt database, and sequences confirmed to be misclassified were either removed completely or taxonomically relabeled. Finally, all sequences <30 amino acids in length were removed and identical sequences were collapsed to a single representative.

Following the length, identity and taxonomy filters, 1,300,994 target protein sequences assigned to 443 distinct species-level taxonomy IDs were left. However, a small subset of viral species contributed the vast majority of protein sequences. For example, 49% of the proteins were from human immunodeficiency virus 1 and 16% were from influenza A virus. To ensure more even representation of viruses within the assay design, the inventors randomly subsampled the overrepresented species, including no more than 2000 and 4000 protein sequences for viruses with RNA and DNA genomes, respectively. Additional protein sequences were allowed for DNA viruses because they often contain larger genomes and proteomes (i.e., more distinct genes). When down-sampling, priority was given to proteins from the Swiss-Prot database, which have been manually reviewed. The final down-sampled target set included 148,215 proteins and 88.78 M amino acids.

In order to optimize potential epitope coverage in as few peptides as possible, a greedy set cover algorithm was utilized in which all potential linear epitopes contained within the target sequences were treated as the “elements of interest” and “sets” were defined as the collection of all potential epitopes contained within a potential peptide probe. Each round, a score was calculated for each potential peptide probe, which corresponded to the sum of the frequencies of each contained epitope within the full target set of proteins, and the highest scoring peptide was added to the design. In the event of a tie, a peptide was randomly chosen from the highest scoring subset. All of the potential epitopes contained within the added peptide were then excluded from the calculation of scores in the next round. This procedure was repeated until a targeted proportion of total epitope diversity was contained within the selected peptides. For the design, the inventors focused on optimizing 9 mer (i.e., 9 amino acids long) epitope coverage using 30 mer peptides.

To reduce the runtime and memory requirements of the algorithm, the target protein sequences were partitioned according to taxonomy prior to running the peptide design algorithm. Subsets of the target proteins were generated by first dividing according to viral family and finally by genus, if the family-level partition contained >500,000 unique 9 mers. Due to the random nature of peptide selection in the event of a tie, the algorithm is not deterministic. Therefore, the inventors independently ran the design for each partition 5-20 times (depending on the size of the partition), and in each case, the inventors selected the result with the fewest number of chosen peptides.

For a subset of species with low numbers of UniProt sequences per annotated protein, unique protein sequences present in GenBank were added to the list of targets. Additionally, for these species and one other with low overall epitope coverage in the set cover design (severe fever with thrombocytopenia syndrome virus, taxID=1933190), peptides were redesigned using a sequence-level (i.e., no alignment) sliding window approach (step=19) in order to optimize epitope coverage. The inventors also included 15 “positive control” peptides, which included epitopes known to be broadly reactive in the human population based on preliminary, unpublished data, and 223 “negative control” peptides designed from an assortment of eukaryotic proteins of exotic species (e.g., coelacanth, coral, great white shark).

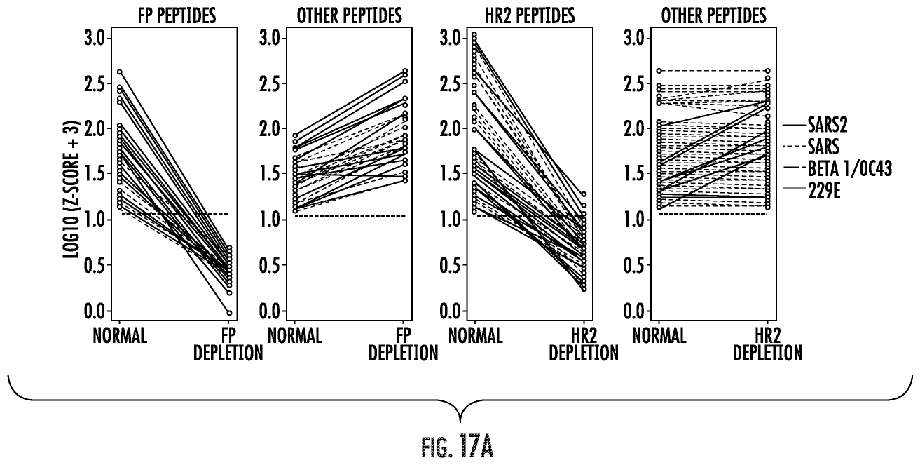

In total, this HV design included 244,000 unique 30 mer peptides, and represents approximately 70% of all potential 9 mer epitopes contained within the target protein sequences. Each of these peptides was represented by a single nucleotide encoding. This design does not contain any peptides derived from SARS-CoV-2 but does contain full proteome coverage of the other six CoVs known to infect humans: Human coronavirus 229E (NCBI taxID: 11137), Human coronavirus NL63 (277944), Human coronavirus HKU1 (290028), Betacoronavirus 1 (694003, includes Human coronavirus OC43), Severe acute respiratory syndrome-related coronavirus (694009, SARS), and Middle East respiratory syndrome-related coronavirus (1335626, MERS) (FIG. 15B).

The second design (SCV2) focused almost entirely on SARS-CoV-2, including high density tiling of peptides across the two most immunogenic SARS-CoV-2 proteins: the spike glycoprotein (S) and the nucleocapsid protein (N). As targets for this design, 2303 SARS-CoV-2 genome sequences downloaded from GISAID were utilized, along with six locally generated sequences. Using these genomes, consensus amino acid sequences for the S and N proteins were first generated. In the design, all of the unique 30 mer peptides contained in these consensus sequences were included, equivalent to a 1-step sliding window approach. Additionally, the inventors used the same epitope-centric set cover design algorithm used for HV in order to capture amino acid-level polymorphisms present within the full set of target genomes. This aspect of the design ensured that 100% of the unique 16 mer peptides present in the S and N proteins from the 2309 SARS-CoV-2 genomes were represented in the design (FIG. 15A). In total, this design included 1550 30 mer peptides from the S protein and 557 30 mer peptides from the N protein. Each of these peptides was represented by three different nucleotide encodings. This design also included a set of 373 control peptides. These controls represent a subset of the HV peptides, which have been determined to be commonly recognized by IgG antibodies in human sera. Therefore, it is expected that some fraction of these controls will be recognized by antibodies in each blood sample tested. Collectively, these peptides were designed from 55 different virus species, including the four endemic human CoVs.

### Peptide-DNA Conjugate Library Assay

To perform serological assays, 5 uL of a 1:10 dilution of serum/plasma in Superblock T20 was added to 0.1 pmol of peptide-DNA conjugate library for a total volume of 10 uL and was incubated at 20° C. overnight. The binding reaction was applied to pre-washed protein G-bearing beads for 15 minutes, after which beads were washed 10 times with 1× PBST. After the final wash, beads were resuspended in 30 uL of water and heated to 95° C. for 5 minutes to elute bound product. Elutions were amplified and indexed using barcoded DNA oligos. Following PCR cleanup, products were pooled, quantified and sequenced on a NextSeq instrument.

### Quantification and Statistical Analysis

PepSIRF v1.3.2, along with custom scripts, was used to analyze the peptide-DNA conjugate HTS data. The data analysis included three primary steps: 1) demultiplexing and assignment of reads to peptides, 2) calculation of enrichment Z-scores individually for each assay and peptide and 3) identification of enriched peptides for each sample based on the consistency of Z-scores and fold-change across replicates.

Demultiplexing and assignment of reads to peptides was done using the demux module of PepSIRF, allowing up to 1 mismatch within each of the index sequences and up to 2 mismatches with the expected DNA tag (90 nt in length). To calculate the Z-scores of the assays and peptides, peptide bins were generated, with each bin containing peptides with similar starting abundance in the peptide-DNA conjugate assay. Each peptide bin contained at least 300 peptides. Starting abundance for each peptide was estimated using buffer-only controls. In total, 8-13 independent buffer-only controls were used to generate the bins for this study. The raw read counts from each of these controls were first normalized to reads per million (RPM) using the column sum normalization method in the norm module of PepSIRF. This was to ensure that independent assays were weighted evenly, regardless of differences in the depth of sequencing. Bins were then generated using the bin PepSIRF module. Prior to Z-score calculation, RPM counts for each peptide were further normalized by subtracting the average RPM count observed within buffer-only controls. This second normalization step controlled for variability in peptide starting abundance within a bin.

Z-scores were calculated using the zscore PepSIRF module, and each Z-score corresponds to the number of standard deviations away from the mean, with the mean and standard deviation calculated independently for the peptides from each bin. It is important that the mean and standard deviation reflect the distribution of unenriched peptides within a bin. Therefore, these calculations were based on the 75% and 95% highest density interval of read counts within each bin for the SCV2 and HV libraries, respectively.

The p_enrich module of PepSIRF was used to determine which peptides had been enriched through the assay. This module identifies peptides that meet or exceed minimum thresholds, in both replicates. For the SCV2 library, a minimum Z-score threshold of 8 was used along with a minimum RPM fold-change of 4. For the HV library, the inventors required a minimum RPM count of 10, a minimum RPM fold-change of 4 and used a 2-tier Z-score threshold, with one replicate needing a Z-score ≥10 and both replicates needing a Z-score ≥6. All of these thresholds were selected to minimize the number of false positive determinations of peptide enrichment based on the analysis of buffer-only negative controls. For both the SCV2 and HV libraries, a range of thresholds was examined using four buffer-only negative controls (analyzed as 6 pairs of replicates), none of which were considered in the creation of the bins. In both cases, the chosen thresholds resulted in only a single enriched peptide being called in ⅙ of the analyzed pairs.

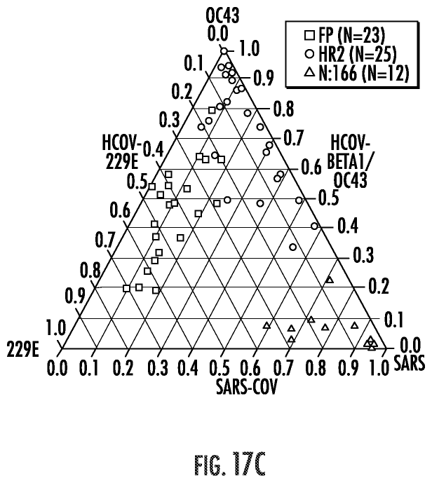

Minimally reactive regions for each epitope were identified as the linear peptide sequences shared by all enriched peptides across convalescent donors. To compare the relative level of reactivity at each epitope to SARS-CoV, HCoV-OC43/Betal and HCoV-229E (FIG. 17C), the inventors first identified all of the peptides designed from each of these species that overlapped the minimally reactive epitope regions. For each epitope, the maximum Z-score for each sample/species pair was identified and then normalized these values by dividing by the sum of the three species-specific Z-scores, with negative Z-scores converted to 0 prior to normalization.

Logistic regression was performed using the glm function in R using log-transformed Z-scores for each of the 6 focal epitopes (peptide with maximum Z-score for each epitope was used) as features to predict convalescent or negative donor status. Cross-validated AUC was calculated by randomly partitioning the data 100 times in 70:30 training:test sets. To quantify correlations between the patterns of reactivity to SARS-CoV-2 epitopes detected by the SCV2 library versus virome-wide peptides in the HV library, the cor. test function in R was used to generate all pairwise Pearson product moment comparisons based on log-transformed Z-scores.

### Identification and Characterization of Antibody Binding Epitopes

In total, the inventors assayed and analyzed 55 COVID-19 convalescent and 69 SARS-CoV-2 negative (both pre- and post-pandemic) serum/plasma samples using the SCV2 and/or HV peptide-DNA conjugate libraries; 96% of the convalescent samples (53/55) and 94% of the negative samples (65/69) were assayed separately with both libraries. Each sample was run in duplicate, and strong signal concordance was observed between technical replicates of the same sera, including those run on different days. Comparative analysis of peptide abundance between serum/plasma and buffer-only negative controls revealed a strong correlation in abundance for the majority of peptides, while a subset of peptides showed distinctly higher relative abundance in each serum/plasma sample (FIG. 15C). These latter peptides are those that have been enriched by binding to serum IgG. To quantify peptide enrichment, Z-scores were calculated for each peptide in each sample. For each peptide, relative abundance was normalized to the corresponding value for the buffer-only negative controls, and this normalized value was compared among peptides with similar abundance in the buffer-only controls. Each Z-score corresponds to the number of standard deviations away from the mean. For a peptide to be considered enriched, the inventors also required a minimum fold-change compared to buffer-only controls and a minimum normalized read count. Z-score, fold-change and read count thresholds were chosen to minimize false negatives based on the analysis of buffer-only controls.

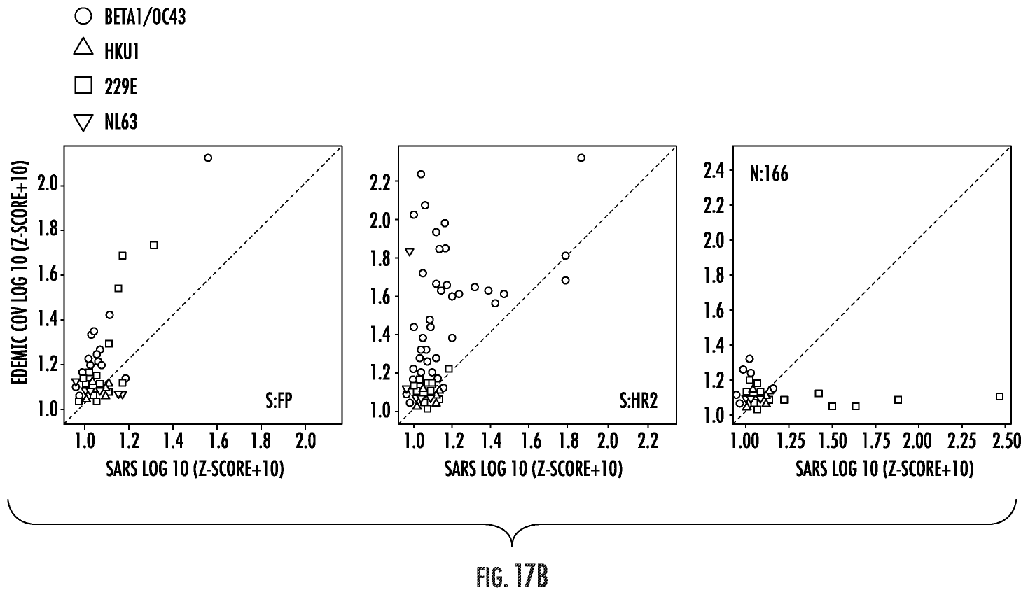

By independently testing reactivity across thousands of potential epitopes, several epitopes with promise for use in both diagnostics and functional characterization assays were identified. In total, the inventors identified IgG reactivity (i.e., peptide enrichment) against 229 and 95 SARS-CoV-2 peptides in convalescent and negative control samples, respectively; 70 of these peptides were enriched in both sample types. The peptides enriched in convalescent samples clustered together into 10 putative epitopes within the S protein and 9 putative epitopes within the N protein (FIGS. 16B and 16C). These epitopes were recognized at a range of prevalences across the sampled population. The six most widely-recognized epitopes—S positions 560-572, 819-824 and 1150-1156 and N positions 166-169, 223-229 and 390-402—were each detected in 13-49% of the convalescent samples tested (median=28.2%, n=55), and all of the convalescent samples with at least one enriched SARS-CoV-2 peptide were reactive to at least one of these six immunodominant regions (FIGS. 16B and 16C).

To explore the diagnostic potential of the six highly recurrent S and N epitopes, the inventors compared the maximum Z-scores per epitope across the full set of convalescent and negative samples (FIG. 16D). Across all six epitopes, the inventors observed an overall shift toward higher Z-scores in convalescent samples, which suggests the presence of additional antibody reactivity at these epitopes that is below the current enrichment thresholds. In fact, at ⅚ of these epitopes, a significant difference in the mean of the Z-score distributions was observed between convalescent and negative samples (t-test, 5560: p=0.001, FP: p=0.036, HR2: p=0.008, N223: p=0.043, N390: p=0.008). To estimate the combined diagnostic performance of these six epitopes, logistic regression models were built using the maximum peptide Z-score for each of the epitopes as features and the donor status (convalescent vs. negative) as the predicted outcome. Cross-validated models each trained on a randomly selected subset of 70% of donors and tested on the remaining 30%, gave a mean area under the curve of 0.92 (FIG. 16E).

For the two epitopes that were detected in the S2 subunit of Spike, structural considerations, as well as previous characterization of related epitopes indicate neutralization potential. A set of 6 recurrent epitopes across the S and N proteins that together exhibit potential for generating an accurate profile of SARS-CoV-2 exposure was also identified. The peptide-DNA conjugate analysis identified a region centered on positions 1150-1156 in the Spike S2 domain (‘HR2’) as the most widely recognized SARS-CoV-2 linear epitope in convalescent donors. The second immunodominant reactivity that was identified in Spike S2 also occurs in a region whose sequence is highly conserved across CoV species. This reactivity is centered on positions 819-824 (‘FP’), which is adjacent to the S2′ cleavage site and overlaps the Fusion Peptide (FP). In addition to the Spike S2 epitopes, some pre-pandemic reactivity to SARS-CoV-2 peptides at the N166 and N390 epitopes was observed.

To more precisely resolve the specificity of the cross-reactive Spike S2 antibodies between the various endemic CoVs, subsets of convalescent donors with strong reactivity in each of the FP, HR2 and N166 epitopes, were selected and then the HV data was used to apportion their relative reactivity across 3 viruses: HCoV-OC43, HCoV-229E and SARS-CoV (FIG. 17C). This was accomplished by normalizing reactivity across the three species using the peptide with the highest Z-score from each. As expected for an epitope without endemic CoV cross-reactivity, the N166 response showed a SARS-dominant profile, with relatively minor reactivities to HCoV-OC43 and HCoV-229E homologs. In contrast, the magnitude of the FP response was primarily dominated by HCoV-OC43 and HCoV-229E, with the relative loadings varying across samples, and a substantially smaller SARS-CoV component, consistent with conservation of this epitope across the three species but indicating stronger recognition of the two endemic homologs. Finally, the HR2 response showed an HCoV-OC43-dominant pattern with a SARS-CoV component that varied across donors, again indicating a response likely shaped primarily by previous exposure to an endemic virus and reflecting the strong conservation of this region between SARS-CoV/SARS-CoV-2 and HCoV-OC43, but not HCoV-229E.

These specific binding and differentially binding epitopes that have been identified and characterized can be further studied as randomized amino acids flanking the identified binding epitopes are incorporated and several binding epitopes are linked together in one polypeptide separated by various spacer sequences, in order to identify higher affinity variants that may additionally exhibit different specificity to the various endemic CoVs. Affinity of these candidate epitopes may also be investigated as candidate peptide binders are paired in single longer peptides, with one peptide ligand on the C-terminus and one on the N-terminus, separated by oligo linkers of different lengths, with each labeled with a unique DNA tag. The peptide-DNA conjugate assay can then be used to identify the combo-peptide with the highest binding affinity.

Binding epitope maturity may also be evaluated as epitopes are modified by complete single residue mutagenesis to identify residues and/or substitutions that might increase binding; by sliding window mutagenesis wherein the N or C terminal portion of the peptide is removed and replaced with random sequences to identify key regions; and by alanine scanning where the original binder is mutated to include alanine or glycine to identify important amino acids in binding.

Through these methods crucial binding epitopes can be identified, characterized, and matured so as to result in epitopes that are the most efficient and/or effective binders that can be applied in future diagnostics and therapeutic applications.

All headings are for the convenience of the reader and should not be used to limit the meaning of the text that follows the heading, unless so specified.

Unless defined otherwise, all technical and scientific terms herein have the same meaning as commonly understood by one of ordinary skill in the art to which this invention belongs. Although any methods and materials, similar or equivalent to those described herein, can be used in the practice or testing of the present invention, the preferred methods and materials are described herein. All publications, patents, and patent publications cited are incorporated by reference herein in their entirety for all purposes.

The publications discussed herein are provided solely for their disclosure prior to the filing date of the present application. Nothing herein is to be construed as an admission that the present invention is not entitled to antedate such publication by virtue of prior invention.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

1. A method of maturing a peptide library to improve binding to a target molecule, the method comprising:
identifying a first peptide having specific binding to the target molecule, the first peptide having an identified threshold z-score;
generating a library of peptide constructs based on the first peptide, the library of peptide constructs comprising:
a peptide construct comprising the first peptide, and
a plurality of peptide constructs comprising variant peptides selected from the group consisting of:
variant peptides that differ from the first peptide by a single point mutation comprising nineteen different peptide variants for each substituted residue of the first peptide;
variant peptides that differ from the first peptide by at least two contiguous residues from either the C-terminus end or the N-terminus end of the first peptide;
variant peptides that differ from the first peptide by a single point mutation and each point mutation is a substitution with alanine; and
variant peptides that differ from the first peptide by a single point mutation and each point mutation is a substitution with glycine;
contacting the target molecule with the library of peptide constructs; and
identifying at least one variant peptide with increased binding to the target molecule compared to the first peptide, wherein the identified at least one variant peptide has a z-score higher than the identified threshold z-score of the first peptide.

2. The method of claim 1, wherein a second peptide is identified as having a z-score higher than the threshold z-score of the first peptide, the method further comprising:
generating a second library of peptide constructs comprising:
a peptide construct comprising the second peptide, and
a second plurality of peptide constructs comprising variant peptides;
contacting the target molecule with the second library of peptide constructs; and
identifying at least one variant peptide from the second library of peptide constructs with increased binding to the target molecule compared to that of the second peptide, wherein the identified at least one variant peptide has a z-score higher than the z-score of the second peptide.

3. The method of claim 2, wherein the second plurality of peptide constructs comprises variant peptides selected from the group consisting of:
variant peptides that differ from the second peptide by a single point mutation and each point mutation is a substitution with alanine; and
variant peptides that differ from the first peptide by a single point mutation and each point mutation is a substitution with glycine.

4. The method of claim 1, wherein at least two variant peptides are identified from the library of peptide constructs with increased binding to the target molecule compared to that of the first peptide, the method further comprising generating a library of peptide constructs that comprise multimers of the at least two variant peptides.

5. The method of claim 1, wherein the library of peptide constructs based on the first peptide comprises at least 10,000 peptide constructs; the library of peptide constructs based on the first and/or second peptide comprise a plurality of negative control peptide constructs; or both.

6-8. (canceled)

9. The method of claim 1, wherein the first peptide comprises a consensus sequence generated from bound peptides, the plurality of peptide constructs comprise variant constructs comprising a core sequence having at least 5 consecutive amino acids from the consensus sequence.

10. (canceled)

11. A method of identifying a peptide with increased specific binding to a target molecule, the method comprising:
providing a first library of peptide constructs;
contacting the target molecule with the first library of peptide constructs to produce at least one peptide construct of the first library of peptide constructs bound to the target molecule and at least one peptide construct of the first library of peptide constructs not bound to the target molecule, wherein a z-score of at least one peptide construct of the first library of peptide constructs not bound to the target molecule is less than a z-score of at least one peptide construct of the first library of peptide constructs bound to the target molecule;
identifying a first peptide from the at least one peptide construct of the first library of peptide constructs bound to the target molecule, wherein the z-score of the selected first peptide is a threshold z-score;
generating a second library of peptide constructs based on the first peptide to identify a higher affinity peptide, wherein the second library of peptide constructs comprises:
a peptide construct comprising the first peptide; and
a plurality of peptide constructs comprising variant peptides selected from the group consisting of:
variant peptides that differ from the first peptide by a single point mutation comprising nineteen different peptide variants for each substituted residue of the first peptide;
variant peptides that differ from the first peptide by at least two contiguous residues from either the C-terminus end or the N-terminus end of the first peptide;
variant peptides that differ from the first peptide by a single point mutation and each point mutation is a substitution with alanine;
variant peptides that differ from the first peptide by a single point mutation and each point mutation is a substitution with glycine; and
variant peptides that comprise at least five consecutive amino acids from the first peptide and at least one of the five consecutive amino acids in the variant peptide is substituted with a different amino acid; and
identifying at least one variant peptide from the second library of peptide constructs with increased binding to the target molecule compared to that of the first peptide, wherein increased binding is indicated by a higher z-score than the identified threshold z-score of the first peptide.

12. A method of identifying a peptide with increased specific binding to a first target molecule and with differential specific binding to the first target molecule and a second target molecule, the method comprising:
providing a first library of peptide constructs;
contacting the first library of peptide constructs with the first target molecule in a first binding assay and with the second target molecule in a second binding assay, wherein the first and the second binding assays produce:
at least one peptide construct of the first library of peptide constructs bound to the first target molecule,
at least one peptide construct of the first library of peptide constructs not bound to the target molecule,
at least one peptide construct of the first library of peptide constructs bound to the second target molecule, and
at least one peptide construct of the first library of peptide constructs not bound to the second target molecule,
wherein a z-score of at least one peptide construct of the first library of peptide constructs not bound to the first or the second target molecule is less than a z-score of at least one peptide construct of the first library of peptide constructs bound to the first or second target molecule;
identifying a first peptide from the at least one peptide construct of the first library of peptide constructs bound the first target molecule, wherein z-score of the first peptide from the first binding assay is a threshold z-score and the z-score of the first peptide in the second binding assay is less than the threshold z-score;
generating a second library of peptide constructs based on the first peptide to identify a peptide with differential specific binding to the first and the second target molecules, wherein the second library of peptide constructs comprises:
a peptide construct comprising the first peptide; and
a plurality of peptide constructs comprising variant peptides selected from the group consisting of:
variant peptides that differ from the first peptide by a single point mutation comprising nineteen different peptide variants for each substituted residue of the first peptide;
variant peptides that differ from the first peptide by at least two contiguous residues from either the C-terminus end or the N-terminus end of the first peptide;
variant peptides that differ from the first peptide by a single point mutation and each point mutation is a substitution with alanine;
variant peptides that differ from the first peptide by a single point mutation and each point mutation is a substitution with glycine; and
variant peptides that comprise at least five consecutive amino acids from the first peptide and at least one of the five consecutive amino acids in the variant peptide is substituted with a different amino acid; and
identifying at least one variant peptide from the second library of peptide constructs with increased binding to the target molecule compared to that of the first peptide, wherein increased binding is indicated by a higher z-score than the identified threshold z-score of the first peptide.

13. The method of claim 12, wherein:
the first target molecule is a tumor cell,
the second target molecule is a normal cell having the same histologic type as the tumor cell, and
the at least one peptide construct with differential specific binding recognizes the tumor cell with higher affinity than the normal cell; and
optionally the first target molecule is a mutant signaling cascade enzyme from a tumor cell,
the second target molecule is a corresponding wild-type signaling cascade enzyme from a normal cell having the same histologic type as the tumor cell, and/or
the at least one peptide construct with differential specific binding recognizes the mutant signaling cascade enzyme with higher affinity than the wild-type signaling cascade enzyme; the mutant signaling cascade enzyme and the wild-type signaling cascade enzyme are protein kinases.

14-15. (canceled)

16. The method of claim 1, wherein the first library of peptide constructs comprises at least 10,000 peptide constructs; the library of peptide constructs based on the first and/or the second peptide comprise a plurality of negative control peptide constructs; or a combination thereof.

17-19. (canceled)

20. The method of claim 11, wherein each individual peptide construct of the first library of peptide constructs comprises:
a peptide portion comprising the first peptide or the variant peptide; and
an identifying nucleic acid portion that identifies the peptide portion.

21. (canceled)

22. The method of claim 20, wherein:
the identifying nucleic acid portion encodes at least 5 randomized amino acids, and
the identifying nucleic acid portion is generated with full nucleotide randomization at the first and second positions of each of at least 5 randomized codons and G/T randomization at the third position to minimize stop codons and maximize synthetic yield, and
further comprising sequencing all or a portion of the identifying nucleic acid portion of the at least one peptide construct of the first library of peptide constructs bound to the target molecule.

23. (canceled)

24. The method of claim 22, wherein the step of sequencing all or a portion of the identifying nucleic acid portion of the at least one peptide construct of the first library of peptide constructs bound to the target molecule comprises amplification and next generation sequencing of the identifying nucleic acid portion; and further comprising selecting at least a second peptide from the at least one peptide construct of the first library of peptide constructs bound the target molecule, the second library of peptide constructs comprises multimers of the selected peptides, wherein the threshold z-score is the highest z-score of the selected peptides and the multimers are dimers comprising a linker between the peptide constructs.

25-29. (canceled)

30. The method of claim 11, further comprising generating a consensus sequence from the peptide portions of the at least one peptide construct of the first library of peptide constructs bound the target molecule, the variant peptides comprise a core sequence comprising at least 5 consecutive amino acids from the consensus sequence.

31. The method of claim 5, wherein the z-score of identified peptides is calculated according to a method comprising:
determining a relative abundance level of each peptide constructs in the library of peptide constructs and/or the second library of peptide constructs;
grouping peptide constructs into bins based on similarity of relative abundance level, wherein each bin comprises at least 300 peptide constructs;
normalizing the relative abundance level of each peptide construct against the average of the relative abundance level of the negative control peptide constructs in the library of peptide constructs and/or the second library of peptide constructs to produce a normalized relative abundance level; and
determinizing a mean and a standard deviation of the normalized relative abundance levels in each bin;
calculating the z-score of each peptide construct based on the mean and the standard deviation of the normalized relative abundance levels in each bin.

32. The method of claim 31, wherein the step of determinizing the mean and the standard deviation of the normalized relative abundance levels in each bin excludes peptide constructs having outlier relative abundance levels; wherein 5% of peptide constructs in each bin are excluded; and/or the mean and the standard deviation of the normalized relative abundance levels in each bin is determine from peptide constructs with normalized relative abundance levels in the 95% highest density interval.

33-34. (canceled)

35. The method of claim 16, wherein the z-score of identified peptides is calculated according to a method comprising:
determining a relative abundance level of each peptide constructs in the first library of peptide constructs and/or the second library of peptide constructs;
grouping peptide constructs into bins based on similarity of relative abundance level, wherein each bin comprises at least 300 peptide constructs;
normalizing the relative abundance level of each peptide construct against the average of the relative abundance level of the negative control peptide constructs in the first library of peptide constructs and/or the second library of peptide constructs to produce a normalized relative abundance level; and
determinizing a mean and a standard deviation of the normalized relative abundance levels in each bin;
calculating the z-score of each peptide construct based on the mean and the standard deviation of the normalized relative abundance levels in each bin; and
optionally, wherein the step of determinizing the mean and the standard deviation of the normalized relative abundance levels in each bin excludes peptide constructs having outlier relative abundance levels; wherein 5% of peptide constructs in each bin are excluded; and/or the mean and the standard deviation of the normalized relative abundance levels in each bin is determine from peptide constructs with normalized relative abundance levels in the 95% highest density interval.

36-38. (canceled)

39. The method of claim 11, further comprising separating the at least one peptide construct of the first library of peptide constructs bound to the target molecule from the at least one peptide constructs of the first library of peptide constructs not bound to the target molecule; wherein the step of separating the at least one peptide construct of the first library of peptide constructs bound the target molecule from the at least one peptide constructs of the first library of peptide constructs not bound to the target molecule further comprises immobilization and/or precipitation of the at least one peptide construct capable of specific binding to the target molecule using a capture agent having specific binding to the target molecule.

40-43. (canceled)

44. The method of claim 39, further comprising immobilizing the peptide portion of the variant construct with increased specific binding to the target molecule or with differential specific binding to the first and the second target molecules to a platform matrix or membrane to produce a diagnostic assay or detection kit the peptide portion of the variant construct is immobilized with an affinity tag/recognition entity interaction; and the affinity tag/recognition entity interaction is selected from the group consisting of polyhistidine/NTA/Ni²⁺, glutathione S-transferase/glutathione, maltose binding protein/maltose, streptavidin/biotin, biotin/streptavidin, and antigen (or antigen fragment)/antibody (or antibody fragment).

45-47. (canceled)

48. A peptide having increased specific binding to a first target molecule and with differential specific binding to the first target molecule and a second target molecule, the peptide identified using the method of claim 11.
